(19) United States
(12) Patent Application Publication
(10) Pub. No.: US 2024/0360458 A1
(43) Pub. Date: Oct. 31, 2024

(54) TARGETED AND SLOW-RELEASE DRUG CONJUGATE COMPOSITION

(71) Applicants: Juewen LIU, Waterloo (CA); Ka-Ying WONG, Waterloo (CA); Yibo LIU, Waterloo (CA); Chau-Minh PHAN, Waterloo (CA); Lyndon JONES, Waterloo (CA); Centre for Eye and Vision Research Limited, Hong Kong (HK)

(72) Inventors: Juewen LIU, Waterloo (CA); Ka-Ying WONG, Waterloo (CA); Yibo LIU, Waterloo (CA); Chau-Minh PHAN, Waterloo (CA); Lyndon JONES, Waterloo (CA)

(21) Appl. No.: 18/596,647

(22) Filed: Mar. 6, 2024

Related U.S. Application Data
(60) Provisional application No. 63/498,498, filed on Apr. 26, 2023.

(51) Int. Cl. C12N 15/115; A61K 9/00; A61K 9/127; A61K 45/06; A61P 27/02

(52) U.S. Cl. CPC C12N 15/115; A61K 9/0051; A61K 9/127; A61K 45/06; A61P 27/02

(57) ABSTRACT

The present invention introduces drug-loaded vehicles conjugated with aptamers, comprising DNA, RNA, or modified nucleic acid sequences designed to selectively and strongly bind to specific target sites on the eye, as well as binding to an ocular device such as a contact lens. Additionally, this innovative approach offers a targeted and sustained drug delivery method, addressing issues such as burst release of drugs, poor affinity between drug and material, and non-targeted drug delivery, while maintaining non-invasiveness and convenience.

### CROSS-REFERENCE TO RELATED APPLICATIONS

The present application claims the priorities from the U.S. provisional patent application Ser. No. 63/498,498 filed Apr. 26, 2023, and the disclosure of which is incorporated herein by reference in its entirety.

### REFERENCE TO SEQUENCE DISCLOSURE

The sequence listing file under the file name “P2729US01_Sequence_listing.xml” submitted in ST.26 XML file format with a file size of 39.7 KB created on Mar. 11, 2024 is incorporated herein by reference.

### FIELD OF THE INVENTION

The present invention generally relates to the field of biomedical engineering, specifically in the development of drug delivery systems related to using DNA, RNA or modified nucleic acid aptamers for ophthalmic applications.

### BACKGROUND OF THE INVENTION

There are several challenges with drug delivery to the eye, such as biological ocular barriers that prevent drugs from reaching the target tissues. Dynamic factors, including blinking and tear drainage, further exacerbate the rapid clearance of drugs from the ocular surface. Recognizing the critical need for enhancing overall ocular drug delivery, strategies to prolong the residence time of drugs on the eye have emerged as a focal point.

One promising avenue involves the utilization of innovative devices such as contact lenses, which play a pivotal role in augmenting drug delivery to the eye. These lenses act as protective shields, enveloping drugs within the post-lens tear film—a layer situated between the lens and cornea. This strategic placement shields drugs from the challenges posed by tear drainage and blinking, thus facilitating an extended residence time on the ocular surface. Nevertheless, the release of drugs from contact lenses often encounters challenges, notably a burst release phenomenon from the material, particularly when there is a lack of strong affinity between the drug and the lens material.

As the demand for advanced drug delivery systems intensifies, driven by a rising prevalence of eye diseases, disorders, and an aging population, there is an imperative to develop strategies that enhance the interaction between drugs and materials, offering better control over drug release dynamics. Aptamers have been studied as a targeting ligand to promote therapeutic effects, but mainly in cancer tissues. The first approved aptamer medication is an anti-VEGF aptamer for inhibiting the growth of abnormal blood vessels in age-related macular degeneration and diabetic macular edema. The second aptamer-based drug is for treating an eye disease called geographical atrophy. A PEGylated aptamer inhibiting platelet-derived growth factor in age-related macular degeneration patients has entered phase 3 clinical trial. Even though the research on aptamer application in the eye is emerging, most of the studies focused on the posterior chamber of the eye, and few studies have been performed ocular drug delivery targeting the eye surface, a major barrier to ocular drug delivery. Additionally, the aptamers selected on cells might not reflect the actual occurrence in vivo, hindering the aptamer application in physiological tissues.

### SUMMARY OF THE INVENTION

Accordingly, the present invention aims to perform tissue-SELEX to select DNA aptamers targeting the surface of the cornea, which could be applied in drug delivery to the cornea.

In a first aspect, the present invention provides a targeted and slow-release drug conjugate composition, which has a carrier conjugated with an aptamer-drug complex. The aptamer-drug complex includes at least one aptamer and at least one therapeutic agent. The aptamer is a short single-stranded DNA or RNA molecule having a nucleotide sequence with a length ranging from 18-60.

In an embodiment, the carrier is a drug-loaded vehicle, such as microparticles, nanoparticles, microcapsules, microspheres, micelles, or liposomes.

In an embodiment, the at least one therapeutic agent includes ocular drugs, such as artificial tears, antibiotics, antivirals, anti-inflammatory drugs, glaucoma drugs, steroids, immunomodulators, or a combination thereof.

In another embodiment, the at least one aptamer is further modified with a linker to achieve high affinity with the ocular device. The linker includes cholesterol, carbodiimide conjugation, thiol-maleimide crosslinking or a combination thereof.

Preferably, the at least one aptamer is cornea-binding aptamer.

In particular, the aptamer-drug complex demonstrates strong binding with one or more ocular regions, characterized by a dissociation constant (K_{d}) of 1-5 nM. Preferably, the K_{d} is 1.2 nm. Their K_{d} values to human corneal epithelial cells (HCECs) were 361 nM and 174 nM, respectively.

In an embodiment, the targeted and slow-release drug conjugate composition is formulated as eye drops, eye ointment or eye gel, eye patches, eye capsules, eye film strips, ocular permeation agents, or ophthalmic gel.

In an embodiment, the single-stranded oligonucleotides include small molecules, proteins, peptides, or nucleic acids.

Additionally, the present invention also provides an ocular device having an aptamer-drug complex attached to one or more ocular regions.

In an embodiment, the ocular device is made from materials including poly methyl methacrylate (PMMA), polyvinyl alcohol (PVA), polyethylene glycol (PEG), dimethyl methacrylate (DMA), hydroxy ethyl methacrylate (HEMA), N-vinyl pyrrolidone (NVP), ethylene glycol dimethacrylate (EGDMA), poly dimethyl siloxane (PDMS), and 3-[tris(trimethyl siloxy)silyl]propyl methacrylate (TRIS).

In an embodiment, the aptamer-drug complex exhibits a high affinity with the one or more ocular regions.

In an embodiment, the one or more ocular regions include an anterior segment comprising conjunctiva, cornea, sclera, iris, ciliary body, lens, or trabecular meshwork.

In another embodiment, the one or more ocular regions include a posterior segment comprising retina, vitreous, choroid, optic nerve head, macula, subretinal space.

In an embodiment, the ocular device includes contact lenses, intraocular lenses.

Additionally, the present invention also provides a non-invasive, target-specific, and sustained drug delivery method for the prevention and/or treatment of ocular diseases or disorders. The method, includes delivering a targeted drug conjugate composition to achieve precise release within one or more ocular tissues. The invention addresses issues such as burst release of drugs from the material, lack of strong affinity between the drug and the material, and non-targeted drug delivery.

The key point to note is that the delivered drugs are not limited to treating eye-related diseases; rather, the eyes can also serve as a delivery site for drugs targeting other types of diseases, such as brain disorders.

In an embodiment, the method achieves release of at least 1-10% of the drug loaded per hour from biological materials.

In an embodiment, the one or more ocular tissues include an anterior segment comprising conjunctiva, cornea, sclera, iris, ciliary body, lens or trabecular meshwork.

In another embodiment, the one or more ocular tissues include a posterior segment comprising retina, vitreous humor, choroid, optic nerve head, macula, subretinal space.

Additionally, the present invention also provides a method for treating/preventing dry eye symptoms, including administering an effective amount of targeted and slow-release drug conjugate composition to a subject in need thereof. The dosage form can be eye drops, eye gel, eye emulsion, eye gel, eye ointment, eye spray, eye hydrogel, or eye suspension, etc.

The present invention introduces novel features not found in current technology. Firstly, it allows for the insertion of dual or multiple aptamers into vehicles, providing the capability to target both biomaterials and specific delivery sites simultaneously. This dual targeting ability enhances the precision and specificity of drug delivery. Additionally, the invention enables sustained release of drugs through the strategic manipulation of aptamer insertion on the vehicle. This feature allows for a controlled and prolonged release of therapeutic agents, contributing to improved efficacy and potentially reducing the frequency of administration. Overall, these distinctive features represent advancements in the field of drug delivery, offering enhanced targeting capabilities and controlled release mechanisms.

### BRIEF DESCRIPTION OF THE DRAWINGS

Embodiments of the invention are described in more details hereinafter with reference to the drawings, in which:

FIG. 1 shows cornea-sclera tissues isolated from pig eyes. Corneal discs of 5 mm in diameter were then cut out by a biopsy punch;

FIG. 2 shows a flow chart of the tissue-SELEX method using pig cornea as a target. A corneal disc was incubated in the DNA library containing an N30 random region flanked by two constant regions for primer binding. The bound DNA was released by adding EDTA, collected and amplified by PCR for the next round of selection;

FIG. 3 shows secondary structures of Cornea-S1 and Cornea-S2 aptamers predicted by Mfold;

FIG. 4 depicts the K_{d} values of Cornea-S1 and Cornea-S2 in HCECs calculated based on flow cytometry;

FIG. 5A depicts flow cytometry data for FAM-labelled Cornea-S1 and Cornea-S2 aptamer in HCECs. FIG. 5B depicts the K_{d} value of S2.2 aptamers to HCECs was calculated based on the FAM fluorescence intensity determined by flow cytometry. FIG. 5C shows the binding of S2.2 aptamer in HCECs. The binding ability of FAM-labelled Cornea aptamers in HCECs. The cell nucleus was stained with DAPI to give blue fluorescence and the green fluorescence was from FAM-labelled aptamers. Scale bar: 20 μm;

FIG. 6 depicts the mean fluorescence intensities of (A) Cornea-S1 and (B) Cornea-S2 in intact cells and disrupted cells;

FIG. 7 shows fluorescence micrographs showing the binding ability of FAM-labelled cornea aptamers in HCECs. Scale bars: 20 m;

FIG. 8 depicts a structure of drug-loaded liposome conjugated with aptamers;

FIG. 9 shows a schematic diagram of cholesterol-conjugated aptamer, loading of CsA in the liposome, and an aptamer-functionalized CsA-loaded liposome;

FIG. 10 depicts the absorbance of free aptamer at 260 nm after aptamer inserted to the liposome;

FIG. 11A depicts a schematic illustration of liposomes specifically delivered to the cornea. FIG. 11B depicts the cornea aptamer binds to a surface protein of the corneal epithelium;

FIG. 12 shows a photograph showing the binding of aptamer-functionalized rhodamine-labeled CsA liposomes to pig cornea. PBS was used as the control. Scale bar: 5 mm;

FIG. 13 depicts the evaluation of K_{d} values of aptamer-conjugated and CsA-loaded liposomes in HCECs. Control means liposomes without any DNA;

FIG. 14A shows fluorescence images of HCECs upon 4-h incubation with aptamer liposomes. FIG. 14B shows fluorescence images of HCECs upon 4-h incubation with aptamer liposomes followed by 20-h incubation in hyperosmolarity medium. Scale bar: 60 m. FIG. 14C depicts quantification of fluorescence intensity of FIGS. 14A-14B. Non-apt: non-aptamer control;

FIG. 15 shows fluorescence images showing time-dependent cellular uptake of liposomes. HECEs were treated with non-aptamer, Corena-S1 or Corean-S2 CsA liposomes for 15 min, 30 min, 1 h, 2 h, or 4 h in a hyperosmolarity medium;

FIG. 16 depicts the cell viability of HCECs upon CsA and liposome treatments for 24 h;

FIGS. 17A-17C depict the gene expression of IL-6, IL-8 and IL-10. Data were normalized with GAPDH and relative to the control;

FIG. 18 shows fluorescence images of treated HCECs incubated with 1 mM fluorescein for 5 min. 4 h followed by 20-h incubation in the culture medium after wash, and quantification of the fluorescent intensity. Scale bar: 60 m;

FIG. 19 depicts representative images of fluorescein staining of the corneal epithelium of rabbits before and after BAC installation, as well as weekly following the initiation of treatments, and the mean fluorescein punctate staining scores were presented as mean±SEM and analysed using One-Way ANOVA analysis;

FIG. 20A shows evaluation of fluorescein punctate staining and tear film. The cornea was divided into five regions for fluorescein punctate assessment. FIG. 20B shows evaluation of fluorescein punctate staining and tear film;

FIGS. 21A-21B depict tear break-up time and tear production in rabbits before and after BAC installation, as well as weekly following the initiation of treatments. FIG. 21C depicts gene expressions of IL-6 and TNF-α in cornea of treated rabbits. The data were normalized to GAPDH and relative to the control; and

FIG. 22A depicts a procedure for applying liposomes to contact lenses. FIG. 22B depicts a process of releasing drug onto the target.

### DETAILED DESCRIPTION

Described herein are various example embodiments, DNA, RNA and modified nuclei acid-aptamer for ocular drug delivery for drugs, therapeutics, and small molecules, biomarkers, and small molecules and associated methods for fabrication thereof and testing in a variety of applications.

One of the main strategies to improve ocular bioavailability of topical eye drops is to increase the retention time of the drug on the eye surface, which is cornea. New techniques for delivering eye drops have emerged, such as prodrugs, cyclodextrins, in situ gels, and nanoparticles. These advancements are aimed at enhancing the absorption and distribution of drugs in both the front and back of the eye, thereby improving their bioavailability. However, active targeting nanomedicine in ocular therapeutics, which could reduce toxicity and adverse side effects, has been less addressed.

Various embodiments for developing aptamers for ocular drug delivery are provided in the present invention. In particular, the present invention provides a targeted and slow-release drug conjugate composition, which has a carrier conjugated with an aptamer-drug complex. The aptamer-drug complex includes at least one aptamer and at least one therapeutic agent. The aptamer is a short single-stranded DNA or RNA molecule having a variable size of nucleotides. In a particular embodiment, it is between 18-60 nucleotides in size. The selected aptamers promote the retention time and therapeutic effect of CsA liposomes under dry eye conditions.

Aptamers are short, single-stranded oligonucleotides that can selectively bind to a wide range of targets, including small molecules, proteins, peptides, nucleic acids (e.g., DNA or RNA molecules), and even entire cells, with high affinity and specificity. It offers several advantages, including faster tissue penetration, low immunogenicity, tolerant to heat stress, ease of modification with different function groups and low-cost synthesis.

The aptamer is composed of two or more sequences designed to bind to a biomaterial and a target ocular site. This dual-binding capability enables the aptamer to provide sustained drug release, as well as increased drug binding to the target sites. Similar to antibodies, the aptamers can be developed to bind to a specific target(s). Aptamer binding can also be improved through modifications to nucleic acids.

The outermost surface of the eyes is cornea, which is composed of corneal epithelial cells. Cornea is the major barrier to drug delivery to eye, which results in low bioavailability and poor efficacy of topical eye treatment. The aptamer selection process utilizes SELEX (Systematic Evolution of Ligands by Exponential Enrichment) to select aptamers that have high specificity and affinity to the targets. SELEX is a complex process and susceptible to artifacts due to dead cells. In some embodiments, capillary electrophoresis-SELEX, random primer-initiated polymerization chain reaction-SELEX, cell-SELEX, microfluidic-based SELEX, magnetic-bead based SELEX, high-throughput sequencing-based SELEX, and machine learning-guided aptamer discovery may be utilized in certain embodiments^{2,3}.

The tissue-SELEX has some differences in steps compared to traditional SELEX, primarily tailored for selection at the tissue level. The tissue-SELEX process of the present invention involves the following steps:
(1) Tissue sample preparation: Tissue SELEX may require the preparation of samples from specific tissues. This might involve the collection, processing, and preparation of tissues to ensure sample purity and integrity.
(2) Sample handling: In the initial stages of SELEX, samples may need different handling to prepare them for interaction with the nucleic acid library. This could involve converting tissue samples into extracts or solutions to facilitate interaction with nucleic acids from the library.
(3) Selection conditions: In tissue SELEX, selection conditions may need adjustment based on the characteristics of the specific tissue environment. This could involve tuning parameters such as temperature, pH, ion concentrations, etc., to ensure effective selection at the tissue level.
(4) Tissue interactions during selection: Tissue SELEX might involve direct interaction between the nucleic acid library and tissue samples to select aptamers with specificity at the tissue level. This may require special experimental procedures to ensure effective interaction between nucleic acids and tissues.
(5) Sample analysis: Following tissue SELEX, further analysis of selected nucleic acids is typically required. This could involve techniques such as real-time or fixed-tissue nucleic acid staining or imaging to assess the binding and specificity of aptamers at the tissue level.

To date, no aptamers have been selected for the surface of the eye or cornea using the tissue-SELEX approach, which is performed on ex vivo tissue-derived structures.

It should be noted that the aptamer selected from various materials can be used in the present invention as long as the selected materials are eye-related or materials for making contact lens and can be used in drug delivery for ocular application.

In some embodiments, the aptamer may be hydrogel aptamer or cornea aptamer. They can bind to both porcine eye and human corneal epithelial cells and possess higher binding affinity to the cells than the previously reported aptamers. For instance, the aptamer may be hyaluronic acid aptamer, collagen aptamer, etc. The binding methods may involve physical adsorption, covalent bonding, or other specific non-covalent interactions, depending on the materials and design employed.

Optionally, the aptamer can be chemically modified with a linker to attach it to the surface of a vehicle. For example, cholesterol can be added to one end of the aptamer, which facilitates its insertion into the lipid bilayer of a liposome.

The carrier is used to capture and deliver drugs, therapeutic agents, or other small molecules. The carrier may be microparticles, nanoparticles, microcapsules, microspheres, micelles, or liposomes.

Liposomes are vesicles composed of lipid bilayers, similar to the natural phospholipid membranes found in cells. Their unique structure allows them to encapsulate both hydrophilic and hydrophobic drugs, making them suitable carriers for a diverse array of therapeutic agents. They enhance drug solubility, protect drugs from degradation, and enable targeted delivery. Liposomes can provide sustained release, reduce side effects, and overcome biological barriers. Their versatility allows for customization in size, surface charge, and composition.

To assemble the composite structure, the process involved integrated at least one drug-loaded liposome with hydrogel aptamers and cornea aptamers selectively bound to the liposome surface.

The binding mechanism encompassed a sequential approach. Firstly, the liposome was prepared with the desired drug payload encapsulated within its lipid bilayers. Subsequently, hydrogel aptamers, designed to enhance stability and provide a responsive matrix, were introduced. The hydrogel aptamers were carefully attached to the liposome through specific non-covalent interactions, which included molecular recognition and physical adsorption. This binding step aimed to establish cohesive integration between the liposome and the hydrogel component. Additionally, cornea aptamers, specialized nucleic acid molecules targeting corneal receptors, were introduced to confer site-specific targeting. The cornea aptamers selectively bound to the liposome-hydrogel assembly, forming a tripartite structure. The overall assembly strategy involved a combination of hydrophobic and hydrophilic interactions, ensuring the stability of the composite structure and enabling targeted drug delivery to the cornea.

In some embodiments, the aptamer has a higher affinity for the target ocular site than the ocular device. For instance, the aptamer binds better to the cornea than the contact lens. This design would enable a higher drug uptake of the aptamer-drug complex into the contact lens material but facilitate the release and binding of this complex to the cornea.

In at least some embodiments, the aptamer may have an affinity to one or more ocular target sites.

In another embodiments, the aptamer is tethered directly to a drug, a therapeutic agent, or a small molecule.

In some embodiments, the at least one therapeutic agent comprises ocular drugs comprising artificial tears, antibiotics, antivirals, anti-inflammatory drugs, glaucoma drugs, steroids, immunomodulators, or a combination thereof. For instance, the possible ocular drug combinations for drug delivery in the present invention are listed in but are not limited to Table 1.

TABLE 1
Possible ocular drug used in this invention

| Class/type of | |
|---|---|
| ocular drug | Drug |
| Artificial tears | Carboxymethylcellulose, Polyethylene glycol, |
| | Propylene glycol, Hydroxypropyl |
| | methylcellulose, and Glycerin |
| Antibiotics | Ciprofloxacin, Moxifloxacin, Tobramycin, |
| | Azithromycin, Ofloxacin, and Erythromycin |
| Antivirals | Acyclovir, Ganciclovir, and Trifluridine |
| Anti-inflammatory | Ketorolac, Diclofenac, Bromfenac, and |
| drugs | Nepafenac |
| Glaucoma drugs | Timolol, Latanoprost, Brimonidine, Dorzolamide, |
| | and Travoprost |
| Steroids | Prednisolone, Dexamethasone, Fluorometholone, |
| | and Loteprednol |
| Inumnunomodulators | Cyclosporine, Tacrolimus, and Pimecrolimus |

In another aspect, the present invention also provides an ocular device having an aptamer-drug complex attached to one or more ocular regions.

In one embodiment, the materials of the ocular device are suitable for aptamer binding. The materials include, but are not limited to, poly methyl methacrylate (PMMA), polyvinyl alcohol (PVA), polyethylene glycol (PEG), dimethyl methacrylate (DMA), hydroxy ethyl methacrylate (HEMA), N-vinyl pyrrolidone (NVP), ethylene glycol dimethacrylate (EGDMA), poly dimethyl siloxane (PDMS), and 3-[tris(trimethyl siloxy)silyl]propyl methacrylate (TRIS)⁴.

In at least some embodiments, the aptamer may have an affinity to the ocular device.

In at least some embodiments, the aptamer may have a higher affinity for the ocular device than the ocular site. This design would enable a slower release of the aptamer-drug complex from the biomaterial, thereby increasing the release duration of the drug.

In one embodiment, the possible ocular sites of interest in the present invention include, but are not limited to, the anterior segment such as conjunctiva, cornea, sclera, iris, ciliary body, lens or trabecular meshwork; and posterior segment such as retina, vitreous humor, choroid, optic nerve head, macula, subretinal space.

### EXAMPLE

### Example 1

### Isolation and Characterization of Aptamer Sequences for Targeted Corneal Drug Delivery

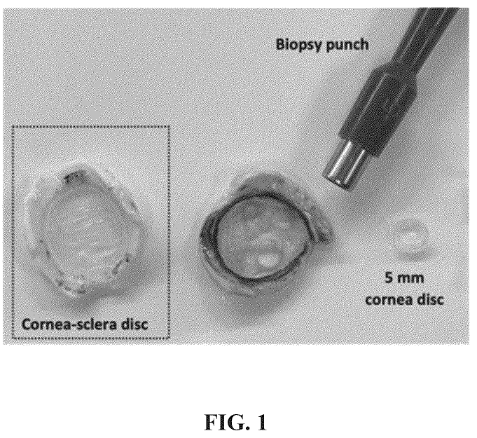

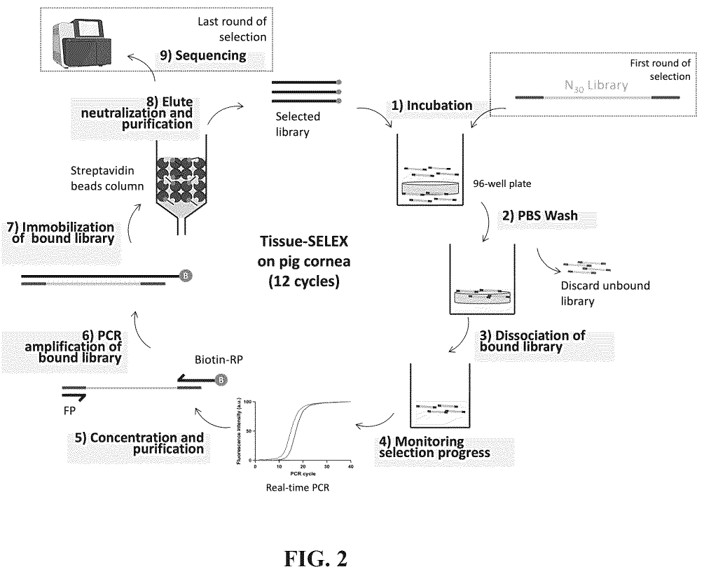

Cornea has a multilayered structure with the outermost layer being corneal epithelial cells. First, the corneas were isolated from pig eyeballs, and they had a hydrogel-like appearance (FIG. 1). Turning to FIG. 2, a DNA library with a 30-nucleotide random region (N30) flanked by two constant primer-binding regions was used for aptamer selection. After incubation of the library (˜10¹⁴ random sequences) with a 5 mm corneal disc and extensive washing, 3 mM ethylenediamine tetra-acetic acid (EDTA) was added to elute the bound DNA. EDTA chelated divalent metal ions, which are important to bind negatively charged DNA to cell surfaces. The eluted bound DNA strands were then amplified by PCR and collected for the next round of selection.

A total of 12 rounds of selection were performed and the last round of the enriched pool was sequenced. Out of the 31,344 sequences obtained, 155 unique sequences with more than two copies were obtained. The 8 most abundant sequences could be divided into a few families, as shown in Table 2 and Table 5. The N30 region were shown in grey. Both families 1 and 2 contained only one sequence, which accounted for 50.8% and 6.19% of the total sequences, respectively. Family 3 contained three highly conserved sequences, which accounted for 4.48% of the total sequences. The number of counts in the other families were less than 1% of the total sequences. Given the complexity of the corneal surface, having abroad distribution of aptamer sequences was expected. In contrast, for small molecule targets, the number of distinct aptamers is often much less.

TABLE 2
The top three families from the cornea-SELEX in the
sequencing results of the round 12 enriched pool

| | Sequence (5′-3′) | Counts |
|---|---|---|
| Family 1 | | 50.8% |
| Cornea-S1 | GACGACGGCAAGGGGAAAGTGGTCGTAATCACGACGGTCGTC | 15,937 |
| Family 2 | | 6.19% |
| Cornea-S2 | GACGACTTATGCTTGGGACCTGATCCGGACTACGGTGTCGTC | 1,941 |
| Family 3 | | 4.28% |
| Cornea-S3 | GACGACGACAAACCAGCGGCTTGTGAGAATGCGTTCGTCGTC | 588 |
| Cornea-S6 | GACGACGACAAACCAGCGGCTTGTGAGAATGCGTTCGCCGTC | 128 |
| Cornea-S7 | GACGACGACAAACCAGCGGCTTGTGAGAATGCGTTCGTCGTC | 60 |

The top two abundance aptamers in the sequencing results were studied, namely Cornea-S1 aptamer (5′-GACGACGGCAAGGGGAAAGTGGTCGTAATCACGACGGTCGTC-Chol) and Cornea-S2 (5′-GACGACTTATGCTTGGGACCTGATCCGGACTACGGTGTCGTC-Chol).

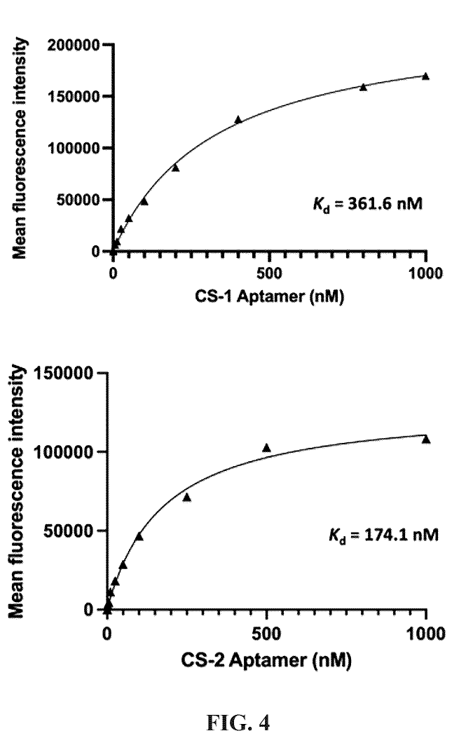

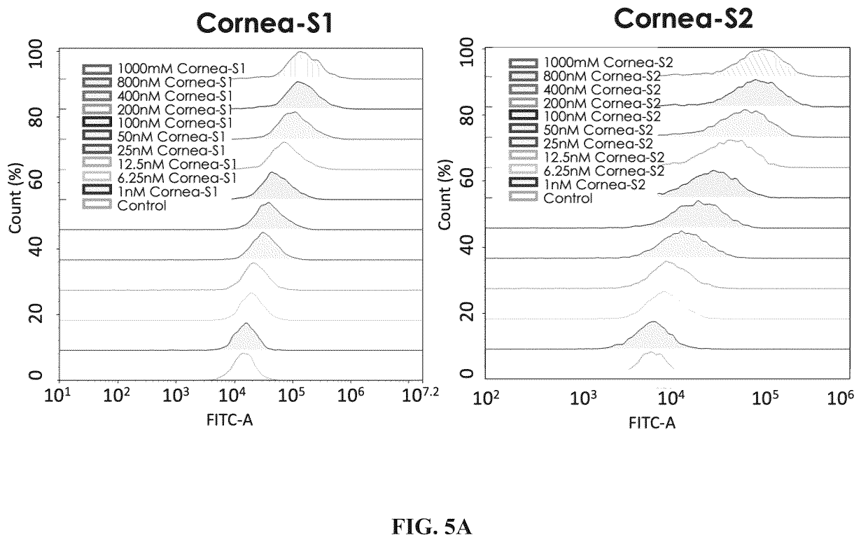

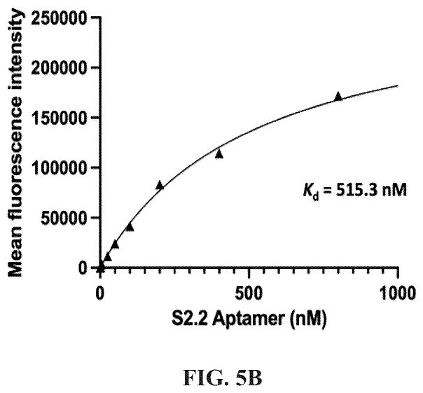

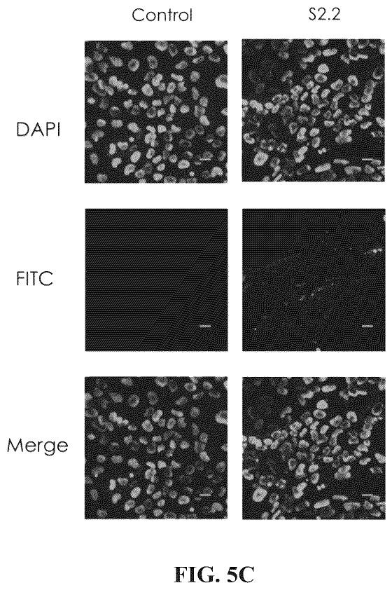

In FIG. 4 and FIGS. 5A-5C, both aptamers showed strong binding to HCECs. Cornea-S1 and Cornea-S2 had dissociation constant (K_{d}) values of 362 nM and 174 nM, respectively. Also, both cornea aptamers had lower K_{d} values than the K_{d} value of the S2.2 mucin aptamer (515 nM) in HCECs, indicating the aptamers selected on cornea tissues had better recognition of eye cells compared to the mucin aptamer. These K_{d} values are not very low for aptamers reported to bind to cells. A potential reason could be that pig corneas were used for the selection, but the cell assays were based on human cells. Nevertheless, the binding affinity can be drastically improved by using polyvalent binding interactions (vide infra).

### Example 2

### Construction of a Dry Eye Disease (DED) Cell Model

To construct a dry eye disease (DED) cell model, HPV-immortalized human corneal epithelial cells (HCECs) (passages 6-10) were maintained in DMEM/F12 medium supplemented with 1% FBS and 1% penicillin/streptomycin at 37° C., 95% humidity, and 5% CO₂. The cell medium was changed every 2 days. To establish an in vitro DED model, sodium chloride was added to the culture medium to increase the osmolarity of the medium by 200 mOsM.

Then, the HCECs were seeded in 96-well plates at 12,000 cells/well and incubated for 24 h. Afterward, HCECs can be treated with vehicle, CsA (0.001% in medium), aptamer or non-aptamer CsA liposomes under serum-free hyperosmolarity medium for 24 h. HCECs cultured in the isosmotic medium with a vehicle were used as a control. Cell viability was measured using MTT assay, following the manufacturer's instructions.

### Example 3

### Preparation of Targeted and Slow-Release Drug Conjugate Composition

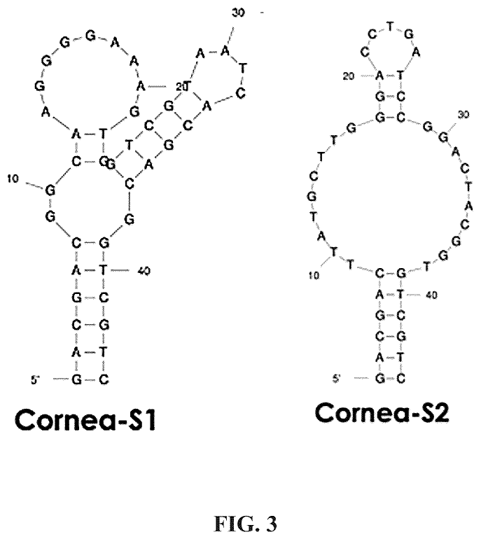

The top two most abundant sequences, Cornea-S1 and Cornea-S2, were further studied since they may have a higher binding affinity or may bind to the most abundant surface molecules on cornea. The secondary structures of Cornea-S1 and Cornea-S2 were predicted by Mfold as shown in FIG. 3.

Due to the large size of cornea tissue, it is difficult to do quantitative binding assays. Thus, the binding of FAM-labelled aptamer to human corneal epithelial cells (HCECs) was tested using flow cytometry and fluorescence microscopy. BSA and salmon DNA were added to the blocking buffer to reduce non-specific binding before the incubation with FAM-labelled aptamers.

HCECs were seeded on glass bottom culture dishes at 10,000 cells/compartment for 24 h. To test the binding of the aptamer to cells, HCECs were incubated with FAM-labelled T30 DNA sequence (negative control), Cornea-S1, or Cornea-S2 aptamers for 30 min. As for liposome uptake in cells, HCECs were incubated with CsA liposomes inserted with non-aptamer, S2.2 aptamer, Cornea-S1 aptamer, or Cornea-S2 aptamer (5 μg mL⁻¹ liposomes) for 4 h with or without 20-h post-incubation in serum-free hyperosmolarity medium. HCECs cultured in the isosmotic medium were used as a control. After incubation, the cells were washed 2 times with ice-cold PBS and were then stained with 1 μg mL⁻¹ Hoechst 33342 in PBS for nucleus staining for 1 min at RT. Fluorescence images were captured at the mid-plane of cells (magnification: 400×) by Nikon Eclipse Ti Inverted Research Microscope. The excitation wavelengths of Hoechst, Rh-PE, and FAM were 361 nm, 560 nm, and 498 nm respectively.

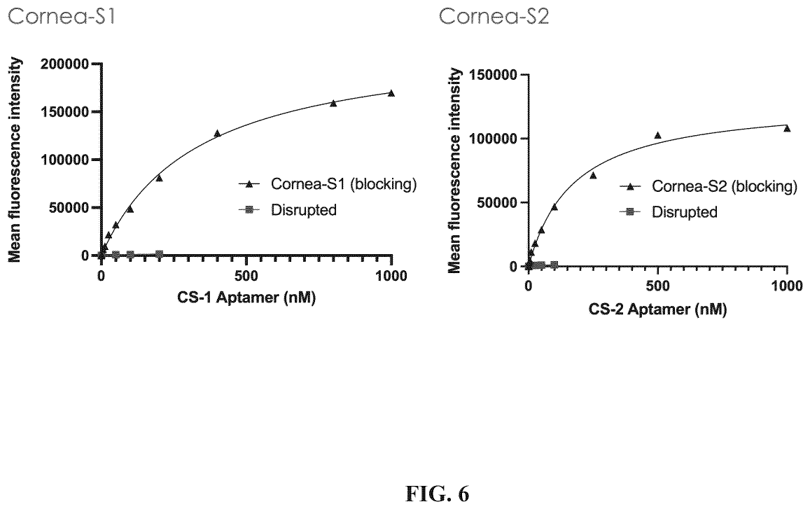

To understand the type of target, the HCECs were treated with trypsin, which degraded surface proteins. Comparing the flow cytometry results between intact cells and trypsin-treated cells, the latter showed significantly lower FAM signals, indicating Cornea-S1 and Cornea-S2 might target cell surface protein of the cornea (FIG. 6).

Furthermore, the binding of FAM-labelled aptamers in HCECs was visualized under a fluorescence microscope. In FIG. 7, no FAM signals were observed in the control and T30 DNA groups, but intensive FAM signals were observed in the Conrea-S1 and Cornea-S2 groups. In particular, the FAM signals in the Corena-S1 group were observably higher than Conrea-S2 and S2.2 aptamer, implying the expression of Cornea-S1 targets might be higher than that of Cornea-S2 targets in HCECs.

The aptamer-functionalized liposomes were synthesized. The liposomes were made of DOPC (1,2-dioleoyl-sn-glycero-3-phosphocholine) and Rhod-PE (1,2-dioleoyl-sn-glycero-3-phosphoethanolamine-N-(lissamine rhodamine B sulfonyl)) at a weight ratio of 99:1 with the CsA drug using the thin-film hydration method.

The modification of liposomes with aptamers could be achieved either during their formation or by conjugating them to synthesized liposomes. When aptamers were conjugated during liposome formation, they were distributed on both the external and internal surfaces, potentially limiting the space for drugs. In this example, post-insertion techniques were employed, introducing cholesterol-tagged aptamers into the lipid bilayer of synthesized liposomes. This approach offered customization benefits for preformed liposomes, allowing better control over aptamer distribution and drug-loading capacity.

Referring to FIG. 8, one or more aptamers were attached to a carrier such as liposome. The carrier was used to capture and deliver at least one therapeutic agent, or other small molecules. The K_{d} of Cornea-S1- or Cornea-S2-functionalized liposomes reduce to 1.2 nM and 15.1 nM respectively, due to polyvalent binding.

Based on dynamic light scattering, the liposomes have a mean particle size of 106 nm and nearly neutral zeta potential. A non-aptamer was used as the negative control sequence and S2.2 aptamer as the positive control sequence. To insert the aptamers with unloaded liposome or CsA liposome, 10 μL of aptamer was mixed with liposome suspension (5 mg mL⁻¹), PBS, and NaCl (pH 7.5, 5 M) at 1:2:2:2 ratio in Milli-Q water to achieve a final volume of 200 μL. After 24-h incubation, the free aptamer was separated from the liposome by ultracentrifugation (Beckman Optima TLX Ultracentrifuge) at 120,000 rpm for 30 min. The concentrations of free aptamer were measured by spectrophotometer (Tecan Spark). The cornea-S1 liposomes or cornea-S2 liposomes were resuspended with 20 μL of PBS (Liposome concentration at 5 mg mL⁻¹). A S2.2 aptamer targeting mucin 1 (S2.2: 5′-GCAGTTGATCCTTTGGATACCCTGGTTTTT-Chol) or a non-aptamer (B2: 5′-ACTTCAACATCTAGCTGGTGG-Chol) was inserted with the CsA liposome as the positive or negative control, respectively.

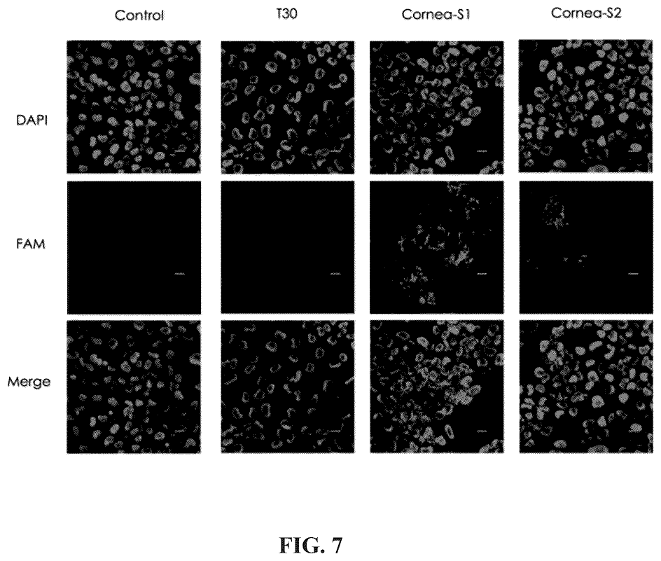

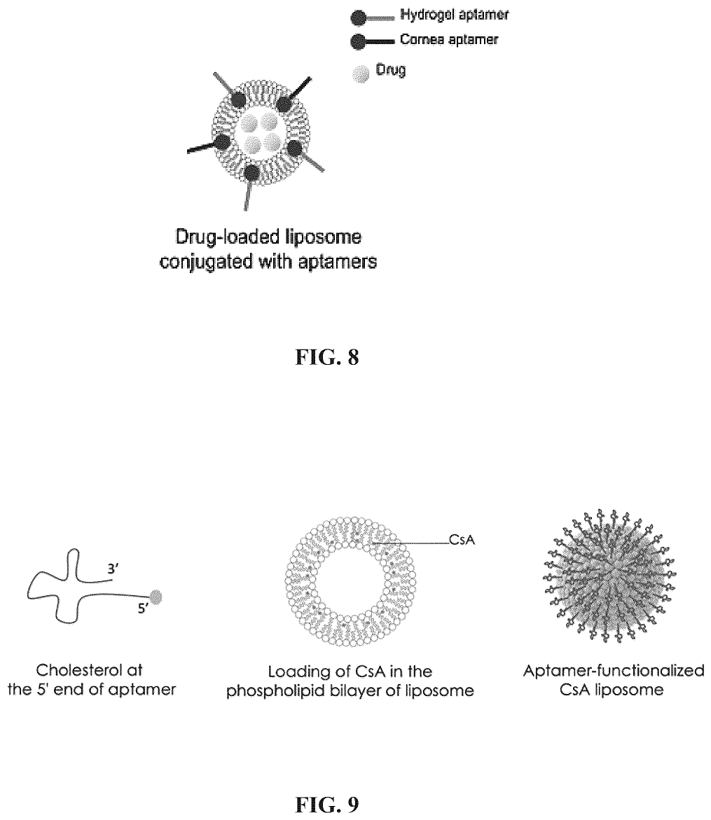

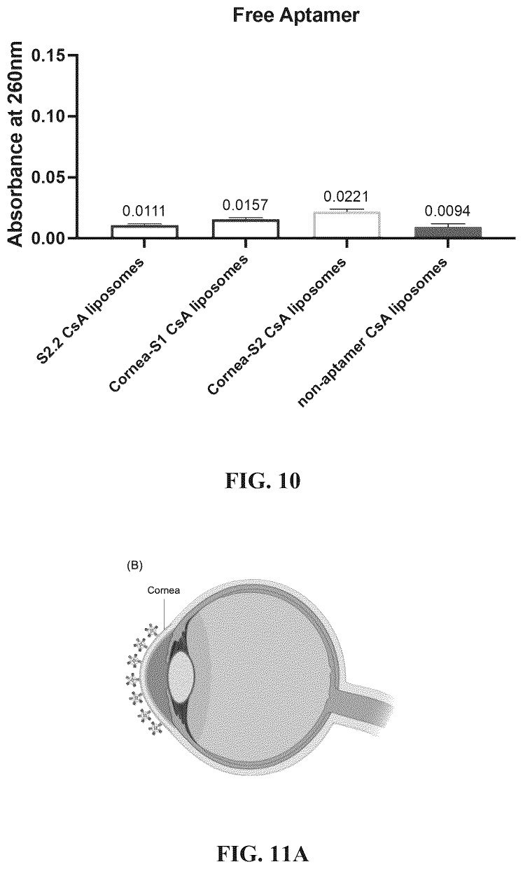

Referring to FIG. 9, non-aptamer, S2.2, Cornea-S1, or Cornea-S2 aptamers were inserted into liposomes through the cholesterol at the 5′ end of the DNA strands. The absorbance measurements at 260 nm were taken for the free aptamer in above four different samples. The values obtained were 0.0111, 0.0157, 0.0221, and 0.0094, respectively (FIG. 10). By using the Beer-Lambert law with a path length of 0.05 cm (measured using a NanoQuant Plate™ in Tecan Spark) and the extinction coefficients of S2.2, Cornea-S1, Cornea-S2, and non-aptamers (200201, 459701, 433301, and 272201, respectively), the number of free aptamers present in a 200 μL reaction were calculated. The results showed that the S2.2 CsA liposomes, Cornea-S1 CsA liposomes, Cornea-S2 liposomes, and non-aptamer CsA liposomes contained 221.7 pmol, 96.5 pmol, 204.1 pmol, and 138 pmol of free aptamers, respectively. After subtracting the number of free aptamers from the initial number of aptamers (1000 pmol), the approximate amount of inserted aptamers present was estimated. The results indicated that the S2.2 CsA liposomes, Cornea-S1 CsA liposomes, Cornea-S2 liposomes, and non-aptamer CsA liposomes contained 779 pmol (78% of the initial amount of aptamer), 904 pmol (90%), 796 pmol (79.6%), and 862 pmol (86.2%) of inserted aptamers, respectively.

FIG. 10 showed the absorbance of free aptamer at 260 nm after aptamer inserted to the liposome. After ultracentrifugation, the concentrations of free aptamer in the supernatant were measured by spectrophotometer, and each liposome had approximately 690 DNA strands.

Furthermore, the size of liposomes was assessed after three months of storage at 4° C. The liposome size remained unchanged (106 nm), indicating stable structure under refrigeration.

### Example 4

### Application of the Drug Conjugate Composition to the Cornea Site

Turning to FIGS. 11A-11B, which illustrated a schematic diagram of cornea aptamer-functionalized liposomes binding to a specific target. The drug-loaded liposomes were conjugated with dual aptamers. These dual aptamers include a hydrogel aptamer designed to target the contact lens and a cornea aptamer targeting the delivery site.

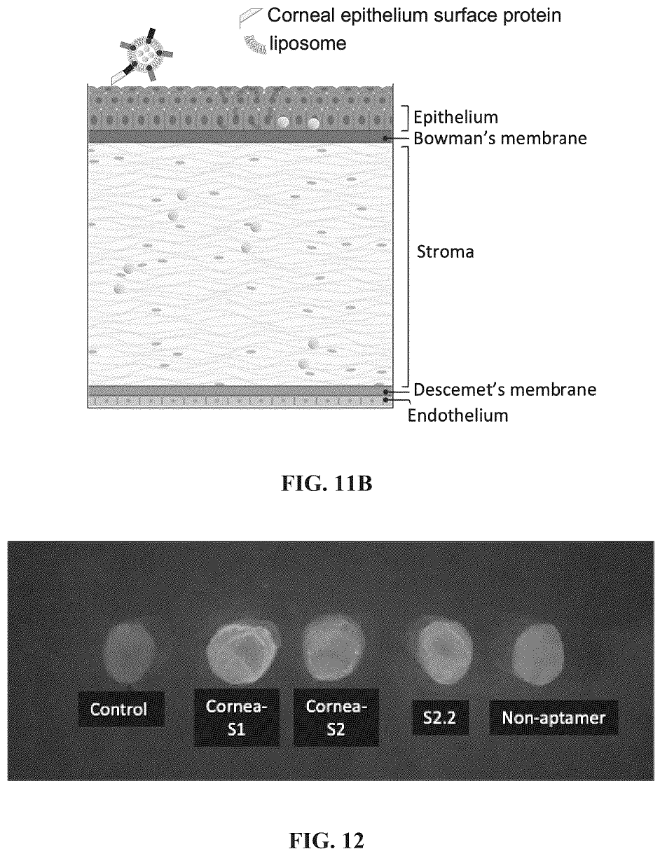

In one embodiment, the Cornea-S1, Corena-S2, or S2.2 Rhod-labeled liposomes were incubated with freshly isolated pig cornea discs followed by extensive wash. In FIG. 12, there was no red fluorescent in the control group (liposomes without DNA attached), indicating low intrinsic red fluorescence of the cornea tissue and low nonspecific adsorption of liposomes to cornea. The Corena-S1 liposomes were found on the surface and mostly at the edge of the pig cornea, implying liposomal binding to the epithelial layer and the cross-sectional area. The S2.2 or Corena-S2 liposomes were found on the cornea surface but with weaker fluorescence. The fluorescence shown in these three groups was apparently greater than that in the non-aptamer group, indicating the aptamers could recognize pig cornea. In addition, since these samples were imaged after extensive washing, these aptamer-functionalized liposomes can achieve a long retention time on the cornea surface.

Following this targeted delivery, the cornea aptamer selectively bound to a surface protein found on the corneal epithelium, as illustrated in FIG. 11B. For instance, a specific example of a surface protein found on the corneal epithelium was E-cadherin, which was a protein involved in cell adhesion, and it played a crucial role in maintaining the integrity of the corneal epithelial layer. Other examples of corneal epithelial proteins may include integrins, claudins, and occludins, which contribute to the structural and functional characteristics of the cornea.

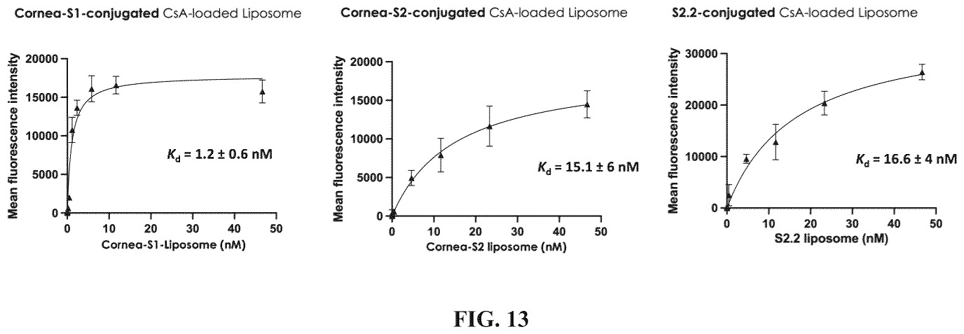

The binding affinity of each aptamer liposome in HCECs was measured by flow cytometry. The cells were incubated with liposomes at room temperature, instead of in a 37° C. incubator, to reduce the internalization of liposomes by endocytosis. In FIG. 13, Cornea-S1-liposomes had the lowest K_{d} values of 1.2±0.6 nM compared to the Cornea-S2 (15±6 nM) or S2.2 (17±4 nM) CsA liposomes. This apparent K_{d} is much lower than that observed in the free aptamers, for example, Cornea-S1 being 300-fold lower, and this was attributed to the stronger polyvalent binding increasing the avidity of the aptamer-functionalized liposomes.

### Example 5

### Therapeutic Efficacy of Targeted and Slow-Release Drug Conjugate Composition

The development of DED is a complex process involving various factors, such as inflammation, tear film instability, and hyperosmolarity. Hyperosmolarity, which is characterized by an excess of solutes in tears, plays a significant role in the pathogenesis of the disease.

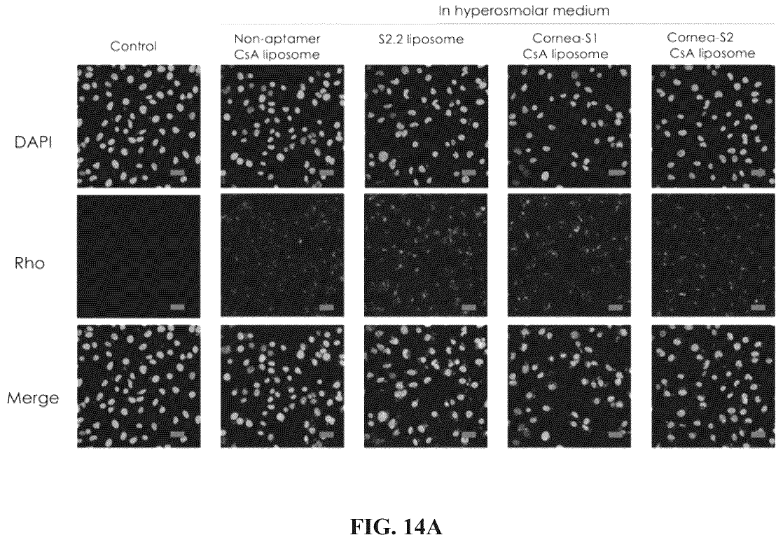

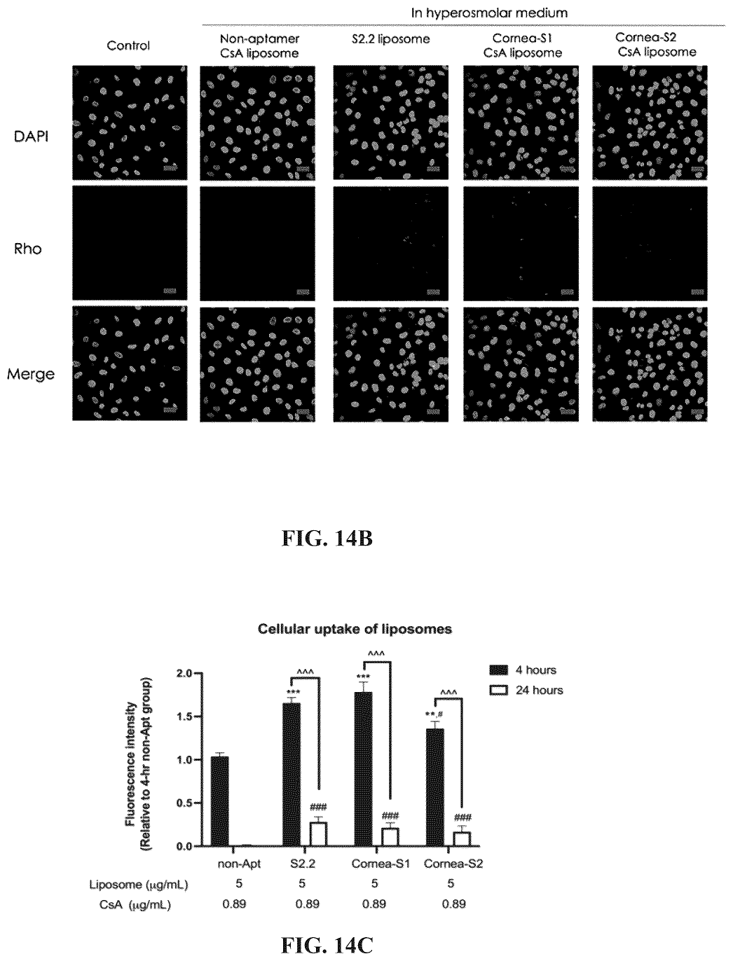

To investigate the effect of the selected cornea aptamers on the retention time and efficacy of CsA liposomes, a non-aptamer was used as the negative control sequence and S2.2 aptamer as the positive control sequence. The CsA-loaded liposomes were tested for cellular uptake and retention in a dry eye HCECs model induced by a high osmolarity medium. In FIGS. 14A and 14C, fluorescent imaging confirmed the cellular uptake of aptamer liposomes in 4 h at 37° C. in HCECs under dry eye conditions. Such results were expected because incubating the cells at 37° C. could allow endocytosis of DNA-functionalized liposome by class A scavenger receptors or in lipid-raft-dependent, caveolae-mediated manner. Most importantly, the fluorescent signals in cells treated with Cornea-S1, Cornea-S2, or S2.2 CsA liposomes were observably and statistically higher than those in the non-aptamer group, indicating aptamers enhanced the cellular uptake of liposomes. Also, the Cornea-S1 liposomes had the highest cellular uptakes in HCECs, potentially due to the better K_{d} values to HCECs. Additionally, liposome retention in cells was assessed by pretreating the cells with liposomes for 4 hours, followed by a 20-hour incubation period in liposome-free medium. The results in FIGS. 14B-14C showed the presence of Cornea-S1, Corena-S2, or S2.2 aptamer CsA liposomes in HCECs after long incubation time, implying aptamer assessed the long retention of CsA liposomes.

In conclusion, in the DED model, the insertion of Conena-S1 or Conrea-S2 aptamers with CsA-loaded liposomes enhanced liposome uptake within 15 minutes and extended retention for at least 20 hours. Ideally, Cornea-S1 or Cornea-S2 enhanced retention extended up to 24 hours.

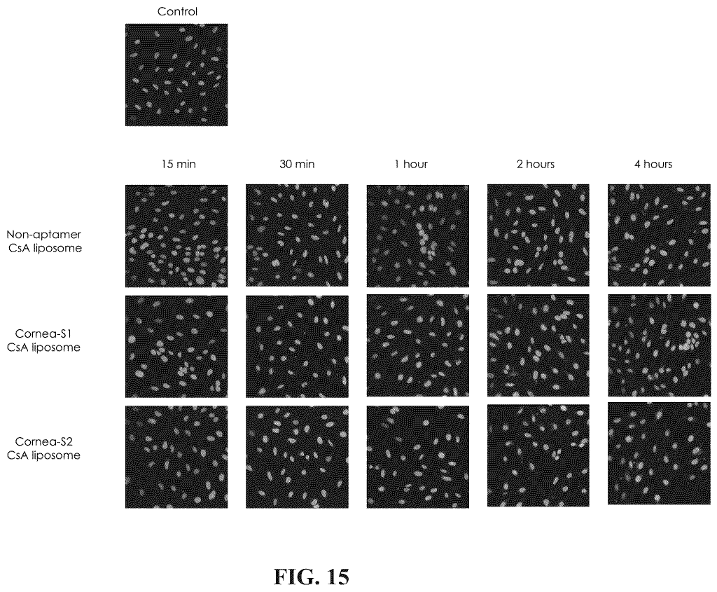

Further investigation was conducted into the kinetics of uptake. The cells were treated with non-aptamer, Cornea-S1, or Cornea-S2 CsA liposomes for 15 min, 30 min, 1 h, 2 h, and 4 h. Referring to FIG. 15, the results indicated the presence of Cornea-S1 and Cornea-S2 aptamers allowed cellular uptake of liposome in 15 min and accumulation of liposome in 4 h in HCECs, while the non-aptamer group required longer incubation time (30 min) to have observable liposome uptake.

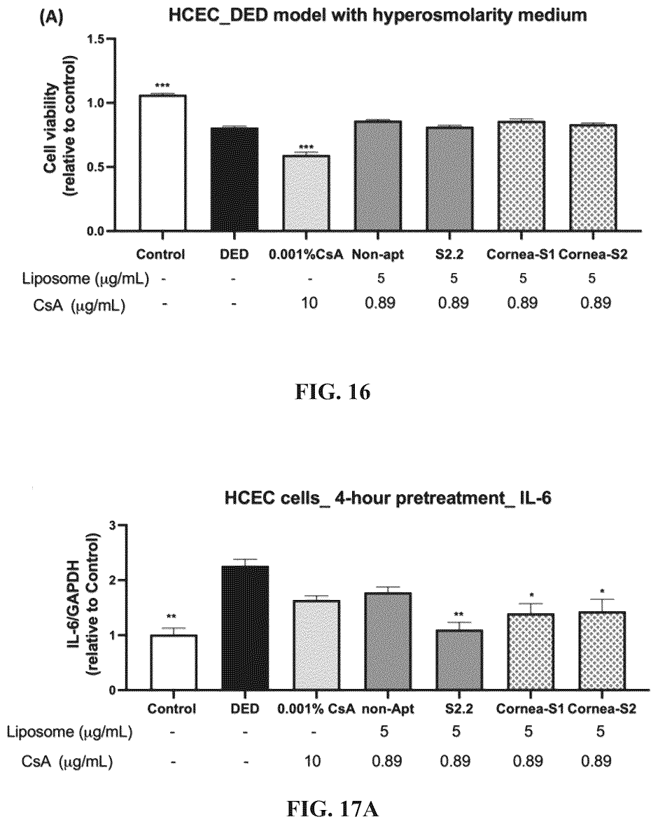

Subsequent investigation focused on evaluating the efficacy of aptamer CsA liposomes (5 μg mL⁻¹) in alleviating dry eye conditions, with comparison to a 0.001% CsA solution. The CsA concentrations in 5 μg mL⁻¹ liposomes and 0.001% CSA in the medium were 0.89 μg mL⁻¹ and 10 μg mL⁻¹, respectively. CsA of 0.001% was chosen based on its therapeutic effects on dry eye cell models as demonstrated previously. The cell viability after 24-h treatment was first evaluated by MTT assay. Referring to FIG. 16, the hyperosmolarity medium significantly induced the death of HCECs compared to the control group. A prolonged 24-h CsA (0.001%) treatment further suppressed cell viability under the hyperosmolarity medium (DED group). Such results implied the cytotoxicity of CsA. The CsA liposomes inserted with aptamers did not cause cell death in HCECs compared to the DED group, suggesting encapsulation of CsA in liposomes reduced CsA cytotoxicity in HCECs.

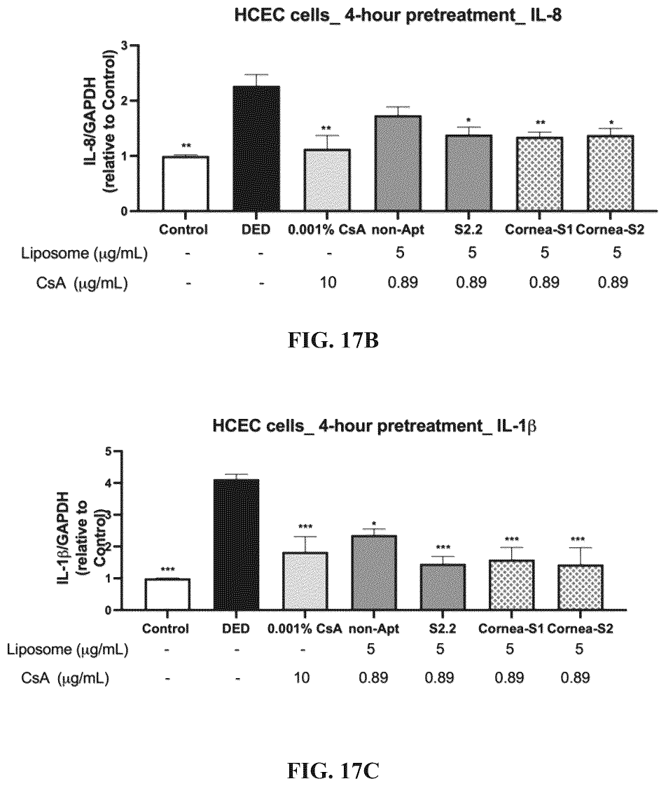

The increase in inflammatory cytokines and chemokines in the tear fluid and the disruption of the tight junction of corneal epithelium are commonly found in dry eye patients. Therefore, the prolonged effect of aptamer liposomes on anti-inflammation and tight junction modulation of liposomes in the dry eye HCECs model was further explored. The cells were treated with liposomes or 0.001% CsA for 4 h in a hyperosmolarity medium followed by 20-h incubation in the treatment-free medium. Referring to FIGS. 17A-17C, the hyperosmolarity medium caused a remarkable increase in the gene expression of inflammatory markers (IL-6, IL-8, and IL-1β) in HECEs compared to the control group. Such increases could be reversed by 0.001% CsA treatment. Cornea-S1 or Cornea-S2 CsA liposomes, which acted like 0.001% CsA, significantly suppressed all inflammatory and tight junction markers in HCECs compared to the DED group. S2.2 CsA liposomes could only abolish hyperosmolarity medium-induced IL-8 and IL-1β gene expressions while non-aptamer CsA liposomes showed no effect on these markers.

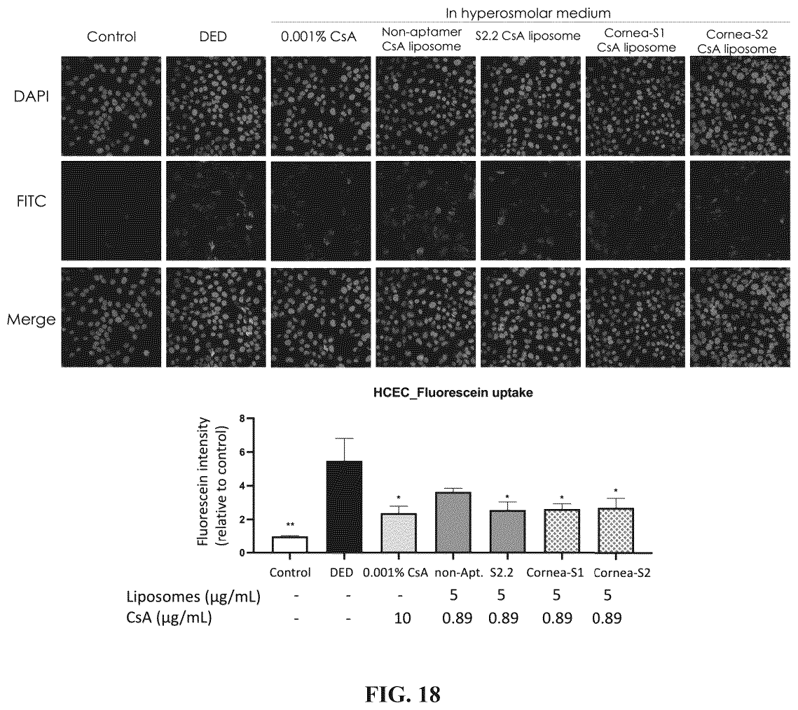

Furthermore, the effect of these liposomes on tight junction modulation was confirmed using the cellular fluorescein uptake assay, a marker dye applied to the evaluation of tight-junctional permeability of epithelial cells. Turning to FIG. 18, a significant increase in fluorescein uptake was observed in HECEs under a hyperosmolarity medium, indicating tight junction disruption in epithelial cells under dry eye conditions. Like 0.001% CsA, S2.2, Cornea-S1 or Cornea-S2 CsA liposomes could significantly reduce the fluorescein uptake in HCECs under hyperosmolality medium while the non-aptamer CsA liposomes did not show a positive effect in this assay. Altogether, the liposomes inserted with aptamers performed better than that with non-aptamer in managing dry eye conditions in HCECs. Also, the use of aptamer-functionalized liposomes for delivery of CsA required a 10-time lower concentration of CsA to achieve a similar effect of CsA alone, which might be resulted from the aptamer-assisted long retention time and uptake of liposomes.

In conclusion, in assessing the anti-inflammatory and tight junction modulation effects of the aptamer-drug complex in the dry eye disease model, Cornea-S1 CsA liposomes were found to sustain corneal integrity and tear break-up time comparably to commercial CsA eye drops, while requiring a lower dosage of CsA. Consequently, the aptamer CsA liposomes achieved similar anti-inflammatory and tight junction modulation effects using only one-tenth of the CsA dosage present in the free drug formulation.

### Example 6

### Dry Eye Treatment in a Rabbit Model

BAC is a commonly used chemical to induce dry eye symptoms, including tear film instability, epithelial cell apoptosis, and inflammation. To further assess the effectiveness of cornea-targeting CsA liposomes in treating DED, an experimental rabbit model of dry eye induced by benzalkonium chloride (BAC) was employed. Various concentrations of BAC were tested, ranging from 0.01% to 1.0%, as well as different frequencies of topical application (two to four times daily) and treatment durations (1-4 weeks), to evaluate their impact on the rabbit ocular surface. The evaluation methods included the Schirmer test, ocular surface staining, conjunctival impression cytology, and microscopic examination.

A 4-week 0.1% BAC treatment could induce stable dry eye condition in rabbits and no severe corneal damage was observed. Thus, a 4-week 0.1% BAC installation followed by a 3-week topical treatment of Restasis® or liposomes was used in the present invention. Corneal fluorescein staining, tear breakup time and tear production were measured before and after BAC installation and weekly throughout the 3-week treatment period. The solutions intended for topical administration were found to have a pH value of 7.41, along with osmolarities of 289-300 mOsms L⁻¹.

Fluorescein is a water-soluble dye that easily permeates and stains the corneal stroma in areas where the epithelium is missing or when the epithelial cells have lax intercellular junctions. This looseness in intercellular junctions is a symptom of DED, which can lead to cell death, damage to corneal barriers, and ultimately, corneal instability.

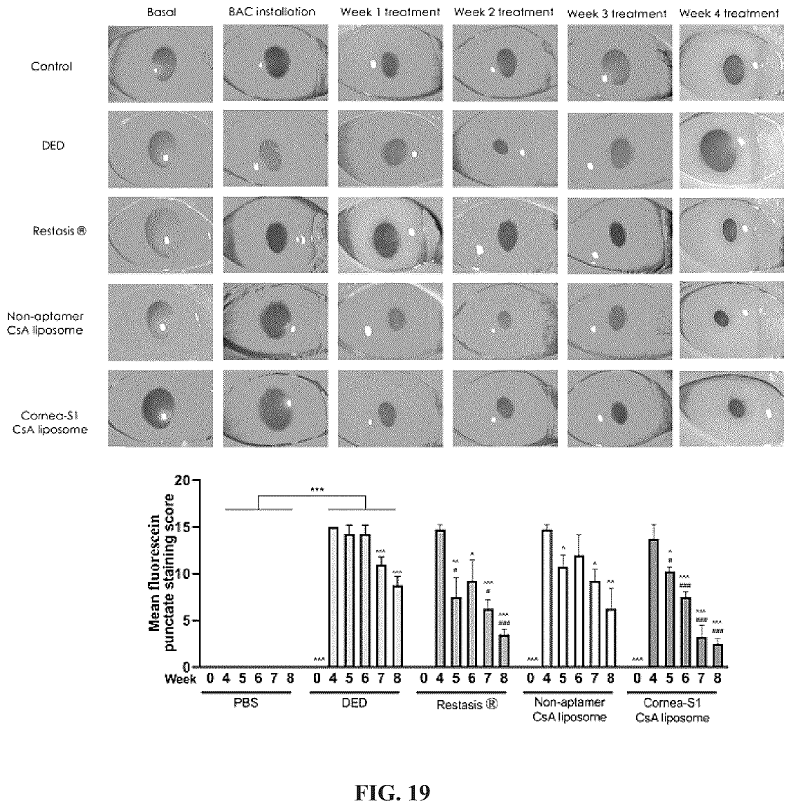

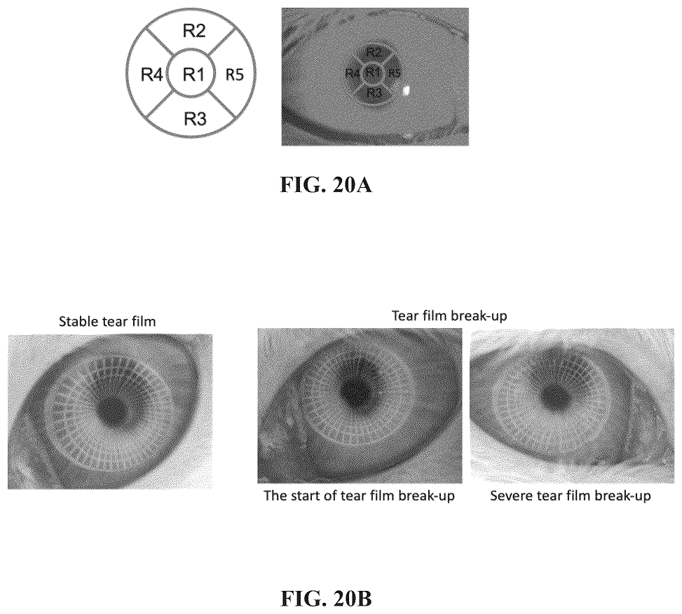

FIG. 19 showed the appearance of the ocular surface under a slit-lamp microscope. No fluorescein staining was observed at the baseline or in the control group throughout the experiment. In contrast, rabbits subjected to the 4-week BAC installation exhibited a significant patch of fluorescein staining on the cornea, indicating the successful establishment of the dry eye model through increased dye permeability and damage to the epithelial layer. The administration of PBS to rabbits with DED did not lead to a significant reduction in fluorescein staining within the initial two weeks, only yielding slight improvement in corneal integrity in regions 2 and 5 in week 3 (FIG. 20A). This outcome strongly suggested the establishment of a stable DED model. In the treatment groups receiving Restasis® or Cornea-S1, the previously pronounced staining was markedly diminished to residual staining or isolated dots following a 2-week treatment period. This observation implied that the impact of Cornea-S1 CsA liposome on preserving corneal integrity was like that of Restasis®, thereby demonstrating a comparable efficacy even at a lower CsA dose encapsulated in the liposomes. Furthermore, in rabbits receiving non-aptamer-bound CsA liposomes, residual fluorescein staining dots persisted on the cornea even after a 3-week period. This observation underscored that the incorporation of corneal aptamers could enhance the effectiveness of CsA liposomes in DED management.

An indicator of evaporative DED is the disruption of tear film homeostasis. DED diagnosis often involves the employment of tear break-up time (TBUT). Visualizing alterations in tear film distribution or corneal surface irregularities can be achieved through the implementation of the tearscope with a grid pattern, where the disruption of the grid indicates the break-up of the tear film (FIG. 20B).

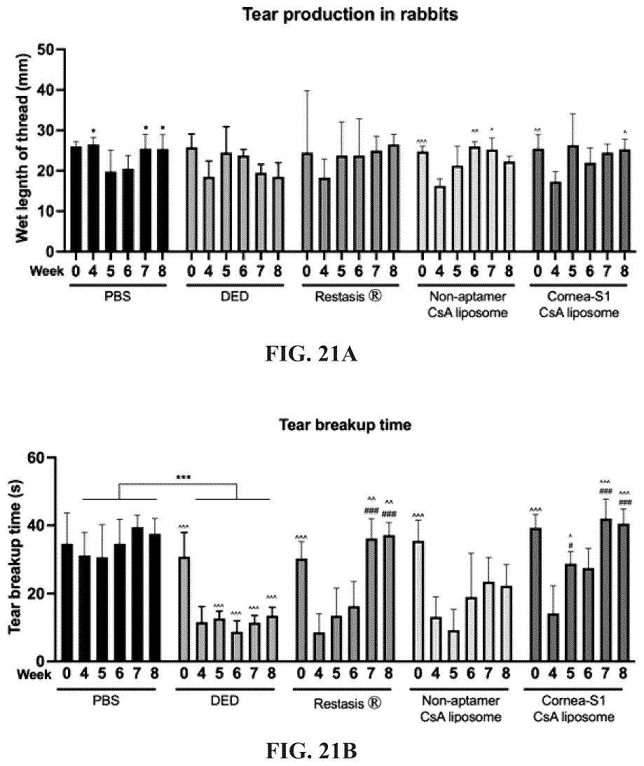

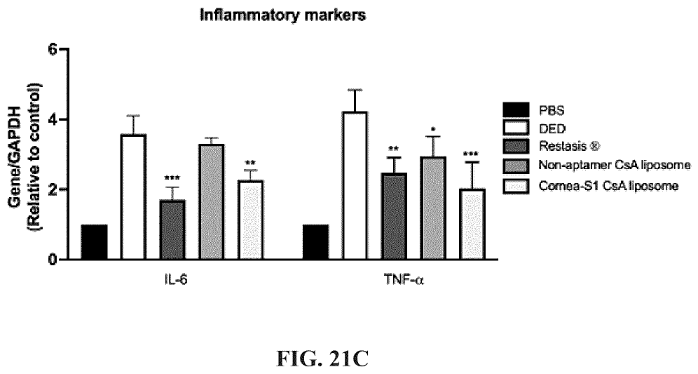

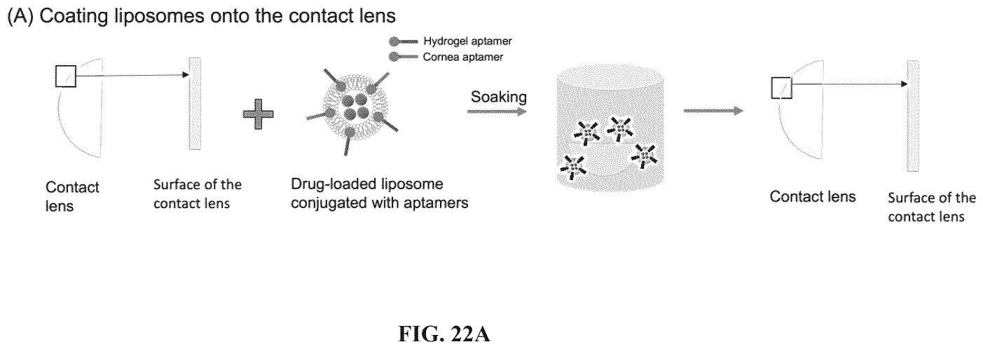

Referring to FIGS. 21A-21C, when compared to the baseline levels, it revealed a notable reduction (approximately 60-70%) in TBUT among rabbits following 4 weeks of BAC installation. This reduction indicated impaired tear film stability. However, this impairment was progressively and significantly alleviated within 3 weeks through the administration of Restasis® or Cornea-S1 CsA liposomes, as evidenced by substantial restoration. Conversely, the DED group or rabbits treated with non-aptamer CsA liposomes displayed limited or negligible restoration.

Importantly, a statistically significant difference in TBUT was noted when comparing rabbits treated with Restasis® or non-aptamer CsA liposomes to those subjected to Cornea-S1 CsA liposomes. A decrease of approximately 25-35% in tear production was observed following the 4-week BAC installation. Such changes were reversed by all three treatments. Although all three treatments led to restoration, statistical significance was not achieved. This lack of significance could potentially be attributed to significant individual variations, stimulated tear production during measurement, or the relatively small sample size. Besides, the gene expression of IL-6 and TNF-α were significantly increased in DED groups, which could be reversed by both Restasis® and Cornea-S1 CsA liposomes while non-aptamer CsA liposomes only reduced TNF-α gene expression. These results indicated the anti-inflammatory effect of CsA could be enhanced when it was loaded in Cornea-S1 liposomes.

### Example 7

### Application of the Targeted and Slow-Release Drug Conjugate Composition to the Surface of the Contact Lens

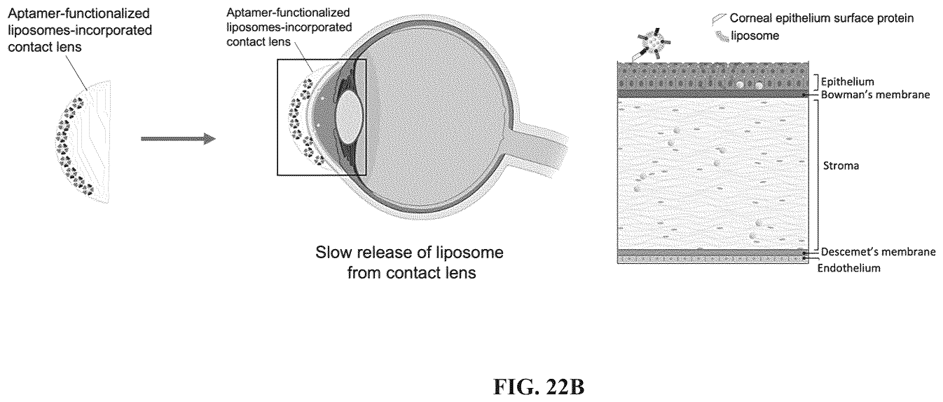

FIG. 22A showed the procedure for applying the aptamer-drug complex to contact lenses. The left side of FIG. 22B showed the coating of aptamer-functionalized, drug-loaded liposomes onto a contact lens. In this process, the liposomes were designed to specifically bind to the target site on the cornea, facilitating the controlled release of the drug from the liposome.

Liposomes may possess specific molecular structures or moieties that exhibit affinity with corresponding portions of the contact lens surface. For instance, one approach involves adding hydrophilic or hydrophobic moieties to the outer layer of liposomes, enabling them to interact with the surface of the contact lens material. Such interactions may include electrostatic attraction, hydrogen bond formation, or other physicochemical mechanisms, allowing liposomes to adhere to the contact lens surface.

Alternatively, specific ligands or biomolecules may be introduced to the outer layer of liposomes, capable of binding with corresponding molecules on the contact lens surface, facilitating the targeted binding of liposomes.

### Example 8-1

### Materials

Porcine eyeballs were obtained from a local market (Highland Packers, Hamilton). Phospholipids were purchased from Avanti Polar Lipids. All aptamers and primers were purchased from Integrated DNA Technologies. Cyclosporine, Fluoromount™ Aqueous Mounting Medium, proteinase K, and deoxyribonucleic acid from salmon sperm were purchased from Sigma-Aldrich. 3K (3K MWCO and 10K MWCO Amicon Ultra-0.5 mL Centrifugal Filters were purchased from Millipore Sigma. Bovine serum albumin was purchased from HyClone. Streptavidin agarose resin and cell culture-related chemicals including medium, serum, and antibiotics were purchased from Fisher Scientific Inc. Culture flasks and Hoechst 33342 solution were purchased from Thermo Fisher. Sodium chloride, Isol-RNA Lysis Reagent, and MTT were purchased from VWR. PBS, iScript™ cDNA Synthesis Kit, Micro bio-spin chromatography columns, and SsoFast EvaGreen supermixes were purchased from Bio-Rad. Glass bottom dishes were purchased from Greiner Bio-One. Milli-Q water was used to prepare all buffers, solutions, and suspensions. All buffers and solutions were prepared with Milli-Q water.

### Tissue-SELEX

Cornea-sclera was isolated from pig eyeballs with dissecting scissors under a dissecting microscope. Corneal discs (5 mm) were cut out using a biopsy punch. Table 3 listed the aptamer selection conditions in each round.

For each round of selection, the DNA library was first heated at 95° C. for 5 min and cooled to room temperature. After washing the corneal disc with PBS twice, the disc was put in a well of a 96-well plate and incubated with 0.5 mL DNA library in PBS with 1 mM Mg²⁺ on an orbital shaker at room temperature for 2 h. The cornea was washed 9 times with PBS with 1 mM Mg²⁺ in new wells (2 mL/wash) on an orbital shaker to remove unbound DNA. The last three washes were collected as a background control. The bound DNA sequences were released by 3 mM ethylenediaminetetraacetic acid (EDTA) for 5 min. The supernatant was collected as the bound DNA. Both background control and EDTA-released DNA were purified by a 3K MWCO centrifugal filter with Milli-Q water 6 times and concentrated to 60 μL under centrifugation at 14,000 rpm 6 times (15 min/time). Real-time polymerase chain reaction (RT-PCR) was then performed to monitor the selection progress. Gel electrophoresis was also performed to confirm the optimal PCR cycles. The bound DNA sequences were amplified using a forward primer and a biotinylated reserve primer.

Subsequently, the PCR products were concentrated and washed using a 10K MWCO centrifugal filter with sterile strand separation buffer (1×PBS, pH 7.5) 10 times under centrifugation at 14,000 rpm for 10 times (10 min/time). To isolate bound DNA sequences, the PCR product was loaded onto a micro chromatography column packed with streptavidin agarose resin. The column was washed 10 times with 500 μL of strand separation buffer followed by the incubation of NaOH for 25 min to elute the bound DNA. An additional 0.2 M NaOH was added to the column followed by the neutralization with 0.2 M HCl. The bound DNA sequences were concentrated and washed with Milli-Q water and separation buffer using a 3K MWCO filter under centrifugation at 14,000 rpm 4 times (15 min/time). The DNA concentration was determined using a Spark microplate reader (Tecan) and subjected to the next round of selection. The bound DNA in round 12 was sequenced by the Illumina method (Table 4). Sequencing results were analyzed with Geneious Prime and Clustal Omega.

TABLE 3

| No. of round | N₃₀ Library (pmol) |
|---|---|
| 1 | 500 |
| 2 | 400 |
| 3 | 300 |
| 4 | 200 |
| 5 | 100 |
| 6 | 100 |
| 7 | 100 |
| 8 | 100 |
| 9 | 100 |
| 10 | 100 |
| 11 | 100 |
| 12 | 100 |

TABLE 4
Sample preparation for DNA sequencing

| Oligo | Sequence (5′→3′) |
|---|---|
| P5-503 | AATGATACGGCGACCACCGAGATCTACACTAT |
| | CCTCTACACTCTTTCCCTACACGACGCTCTTCC |
| | GATCTACAGTCATCATAGAGTCGGGACG |
| P7-704 | CAAGCAGAAGACGGCATACGAGATGCTCAGGA |
| | GTGACTGGAGTTCAGACGTGTGCTCT TCCGA |
| | TCTGGAGGCTCTCGGGACGAC |

The enriched pool from round 12 was used as the DNA template and was subjected to another PCR reaction using forward primer (P5-503) and reverse primer (P7-704) containing unique index sequences were used. Agarose gel was used to purify the PCR products subjected to gel extraction using a small DNA fragment extraction kit (IBI Scientific). The concentration of the purified DNA was quantified. The samples were submitted to McMaster University Genomics Facility for Illumina sequencing.

### Example 8-2

### Animal Experiment

Ten male New Zealand White rabbits, weighing between 3 to 3.5 kg each, were procured from Charles River Laboratories. They were housed within the Centralized Animal Facility at the University of Waterloo, where they were subjected to a 12-hour cycle of alternating light and darkness. The temperature was maintained at 23° C.±2° C., with a relative humidity of 60%±10%. The entire experimental procedure was conducted by ethical guidelines and received approval from the Animal Care Committee under Application No. 44737. Upon a 2-week acclimation period, eight rabbits were chosen at random to induce stable dry eye disease. This was achieved through the topical application of 0.1% benzalkonium chloride (BAC) at a dosage of 20 μL per installation. This protocol was repeated twice a day (at 10 a.m. and 5 p.m.) for the subsequent 4 weeks. The remaining two rabbits constituted the control group and received PBS through a similar administration schedule. Following this phase, all rabbits were randomly divided into five groups, each comprising two rabbits (4 eyes). Over the course of 3 consecutive weeks, the rabbits in each group received the designated treatments twice daily (at 9 a.m. and 5 p.m.) as follows: 1) Control Group: 20 μL of PBS; 2) DED Group (Model Group): 20 μL of PBS; 3) Positive Control Group: 20 μL of Restasis® 0.05% CsA eye drops; 4) Negative Control Group: 20 μL of sterile non-aptamer CsA liposomes (0.5 mg mL⁻¹) and 5) Experimental Group: 20 μL of sterile aptamer CsA liposomes (0.5 mg mL⁻¹). In the Restasis® group, each application contained 10 μg of CsA, while in the liposomes group, the amount was reduced to 0.89 μg per application.

### Example 8-3

### Methods

### Determination of Cell Membrane Permeability

HCECs were seeded on glass bottom culture dishes at 10,000 cells/compartment for 24 h. The cells were treated the same as in real-time PCR. After treatment, the cells were rinsed 2 times with PBS. The membrane integrity was studied using a fluorescein uptake assay. The cells were incubated with fluorescein (1 mM) in the medium for 5 min followed by the 1 μg mL⁻¹ Hoechst 33342 in PBS for 1 min at RT. The fluorescein uptake was captured as mentioned. The excitation wavelength of Hoechst and fluorescein were 361 nm and 498 nm, respectively. The overall intensities of the fluorescent signals were also quantified using the corresponding Nikon system.

### Flow Cytometry

To investigate the binding affinity of FAM-labelled aptamer or aptamer liposomes to the cells, HCECs were harvested using a scrapper or detached from the flask by dissociation solution (0.25% trypsin and 0.1 mg mL⁻¹ proteinase K). The cells were then incubated with blocking buffer (1% BSA, 0.1 mg mL⁻¹ Salmon sperm DNA in PBS) for 30 min at room temperature. After washing with PBS under centrifugation, 3×10⁵ cells were incubated with pre-heated Cornea-S1 or Cornea-S2 aptamers or aptamer liposomes at various concentrations in PBS for 30 min at room temperature. After incubation, the cells were rinsed three times with 500 μL of PBS and then resuspended in 200 μL of PBS. The fluorescence signal was analyzed with a NovoCyte Flow Cytometer (Agilent Technologies). The equilibrium dissociation constants (K_{d}) value of each aptamer or aptamer liposome was obtained by GraphPad Prism 9 according to

$$Y = Bmax * {X ( {K}_{d} + X )}^{- 1} .$$

### Real-Time PCR of Cornea Tissue

The cornea of rabbits was collected and homogenized. RNA was extracted from the cells using Isol-RNA Lysis Reagent, and reverse transcription was performed using the iScript™ cDNA Synthesis Kit according to the manufacturer's instructions. The expression levels of target genes, including interleukin-6 (IL-6), Tumor Necrosis Factor Alpha (TNF-α) and glyceraldehyde-3-phosphate dehydrogenase (GAPDH), were measured using SsoFast EvaGreen with specific primer pairs (Table 5). The gene expression was normalized to GAPDH. The sequences have more than 0.1% counts in the sequencing results were analysed and grouped in 6 families after sequence alignment.

TABLE 5

| | Sequence (5′-3′) | Counts |
|---|---|---|
| Family 1 | | 50.8% |
| Cornea-S1 | GACGACGGCAAGGGGAAAGTGGTCGTAATCACGA | 15,937 |
| | CGGTCGTC | |
| Family 2 | | 6.19% |
| Cornea-S2 | GACGACTTATGCTTGGGACCTGATCCGGACTACGG | 1,941 |
| | TGTCGTC | |
| Family 3 | | 4.28% |
| Cornea-S3 | GACGACGACAAACCAGCGGCTTGTGAGAATGCGT | 588 |
| | TCGTCGTC | |
| Cornea-S6 | GACGACGACAAACCAGCGGCTTGTGAGAATGCGT | 128 |
| | TCGCCGTC | |
| Cornea-S7 | GACGACGACAAACCAGCGGCTTGTGAGAATGCGG | 60 |
| | TCGTCGTC | |
| Family 4 | | 0.91% |
| Cornea-S4 | GACGACAAGTTCCCCGGCAGGGCCATTGTGAGAA | 285 |
| | AACGTCGTC | |
| Family 5 | | 0.55% |
| Cornea-S5 | GACGACAGCGAGCCGGAGGGAATGAGAACGAGT | 174 |
| | GGAGTCGTC | |
| Family 6 | | 0.11% |
| Cornea-S8 | GACGACTCTACTTTACATGGTCAATGGGCGTGCGG | 36 |
| | TGTCGTC | |

### Real-Time PCR of HCECs

HCECs were seeded in 6-well plates at 250000 cells/well and incubated for 24 h. Afterward HCECs were treated with CsA (0.001% in medium), aptamer or non-aptamer CsA liposomes (5 μg mL-1 liposomes) under serum-free hyperosmolarity medium for 4 h. After incubation, the cells were further incubated in a serum-free hyperosmolarity medium for 20 h. The reverse transcription and real-time PCR were performed as described previously. The sequences of specific primer pairs were listed in Table 6.

TABLE 6

| Oligo | Sequence (5′→3′) |
|---|---|
| IL-1ß forward primer | CCTGTCCTGCGTGTTGAAAGA |
| IL-1ß reverse primer | GGGAACTGGGCAGACTCAAA |
| IL-6 forward primer | CACAGACAGCCACTCACCTC |
| IL-6 reverse primer | TTTTCTGCCAGTGCCTCTTT |
| IL-8 forward primer | TTTCAGAGACAGCAGAGCACACAA |
| IL-8 reverse primer | CACACAGAGCTGCAGAAATCAGG |
| GAPDH forward primer | GAAGGTGAAGGTCGGAGTC |
| GAPDH reverse primer | GAAGATGGTGATGGGATTTC |
| Library | GGAGGCTCTCGGGACGACN30GTCGTCCCGACT |
| | CTATGATGACTGT |
| Column-binding strand | GTCGTCCCGAGAGCCATA-/3Biosg/ |
| SELEX forward primer | GGAGGCTCTCGGGACGAC |
| SELEX reverse primer | ACAGTCATCATAGAGTCGGGACG |
| SELEX Biotin-reverse primer | /5Biosg/ACAGTCATCATAGAGTCGGGACG |
| Cornea-S1-cho | GACGACGGCAAGGGGAAAGTGGTCGTAATCAC |
| | GACGGTCGTCTTTTT-Chol |
| Cornea-S2-cho | GACGACTTATGCTTGGGACCTGATCCGGACTAC |
| | GGTGTCGTCTTTTT-Chol |
| S2.2-cho | GCAGTTGATCCTTTGGATACCCTGGTTTTT-Chol |
| B2-cho | ACTTCAACATCTAGCTGGTGG-Chol |
| FAM-Cornea-S1 | GACGACGGCAAGGGGAAAGTGGTCGTAATCAC |
| | GACGGTCGTC/36-FAM/ |
| FAM-Cornea-S2 | GACGACTTATGCTTGGGACCTGATCCGGACTAC |
| | GGTGTCGTC/36-FAM/ |
| Biotin-Cornea-S1 | /5Biosg/GACGACGGCAAGGGGAAAGTGGTCGTA |
| | ATCACGACGGTCGTC |
| Biotin-Cornea-S2 | /5Biosg/GACGACTTATGCTTGGGACCTGATCCGG |
| | ACTACGGTGTCGTC |
| IL-6 Forward | GTCTTCCTCTCTCACGCACC |
| IL-6 Reverse | TGGGCTAGAGGCTTGTCACT |
| TNF-α Forward | GTCTTCCTCTCTCACGCACC |
| TNF-α Reverse | TGGGCTAGAGGCTTGTCACT |

### Fluorescein Staining

5 μL 100 fluorescein was dropped onto the ocular surfaces of the rabbits under sedation by an intramuscular injection of acepromazine (1 mg kg⁻¹ body weight). The eyelids were blinked manually 2-3 times and then held open to distribute the fluorescein. After 5 min, the eye was flushed with 2 mL sterile saline. The distributed fluorescein was observed with a slit lamp. Briefly, the cornea was divided into five areas (FIG. 20A, central, superior, nasal, inferior, and temporal). The severity of corneal fluorescein staining was graded on a scale from 0 to 3. The scores from 5 zone were then added up with a maximum score of 15.

### Tearscope Analysis for Tear Break-Up Time (TBUT) and Tear Production

Rabbits were sedated by the aforementioned procedure. TBUT was evaluated utilizing a handheld EasyTear® VIEW+ Tearscope with a grid pattern, in conjunction with the S4 OPTIK HR Elite Mega Digital Vision system. To simulate a blink, the eyelids were gently closed, initiating the TBUT assessment immediately upon the re-opening of the eye. This measurement was repeated thrice to ensure accuracy, and an average value was calculated for analysis.

The aqueous tear production was measured with Schirmer Tear Strips (SPORTS WORLD VISION). The strips were placed inside the margin of the nictitating membrane of the lower eyelid of the rabbit for 5 min and the wetting length of the strip was measured as an indication of tear production.

Statistical analysis

For MTT, RT-PCR, flow cytometry, and cell cellular uptake assays, the data were reported as mean±SEM. Inter-group differences were analyzed by one-way ANOVA with Tukey's as a post hoc test. A value of P<0.05 was considered statically significant. All graphs in this study were plotted by using GraphPad Prism Version 9.0.

In summary, the present invention has the advantages of sustained release of drugs from materials, strong interaction between the drug and the material, and targeted drug delivery. Dual or multiple targeting drug delivery in contact lenses presents a unique opportunity to improve therapeutic outcomes, convenience, and patient compliance, and has the potential to transform the way for treating eye conditions.

The contribution of the present invention involves conducting tissue-SELEX to identify new aptamers for the cornea, demonstrating a comparable targeting effect to the mucin aptamer. The outcomes of this research provide convincing evidence supporting the feasibility of targeted corneal treatment using aptamers as an effective approach for addressing DED. The trends observed in these findings underscore the necessity for further investigations, including localized and systemic delivery of CsA, distribution of aptamer-functionalized liposomes on the ocular surface, and the immune response elicited by aptamer-functionalized liposomes within the body.

This technology aims to provide targeted and sustained drug delivery to the eye, addressing the unmet needs of patients with various eye conditions, such as dry eye syndrome, glaucoma, eye infections, and retinal diseases.

### Definition

Various processes will be described below to provide an example of at least one embodiment of the claimed subject matter. No embodiment described below limits any claimed subject matter, and any claimed subject matter may cover processes or systems that differ from those described below. The claimed subject matter is not limited to processes or systems having all of the features of any process or system described below or to features common to multiple or all of the processes or systems described below. It is possible that a process or system described below is not an embodiment of any claimed subject matter. Any subject matter that is disclosed in a process or system described below that is not claimed in this document may be the subject matter of another protective instrument, for example, a continuing patent application, and the applicants, inventors, or owners do not intend to abandon, disclaim or dedicate to the public any such subject matter by its disclosure in this document.

It should be noted that terms of degree such as “substantially”, “about” and “approximately” as used herein mean a reasonable amount of deviation of the modified term such that the end result is not significantly changed. These terms of degree may be construed as including a certain deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made if the end result is not significantly changed.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The term “aptamer”, in the context of the present invention, is related to single stranded nucleic acid chains that adopt a specific tertiary structure that enables them to bind to molecular targets with high specificity and affinity, comparable to that of monoclonal antibodies, through interactions other than classical Watson-Crick base pairing, such as electrostatic and ionic interactions.

The term “nucleic acid”, in the context of the present invention, refers to any type of nucleic acid, such as DNA and RNA, and variants thereof, such as peptide nucleic acid (PNA), locked nucleic acid (LNA), as well as combinations thereof, modifications thereof, that include modified nucleotides, etc. The terms “nucleic acid” and “oligonucleotide” and “polynucleotide” are interchangeably used in the context of the present invention. Nucleic acids can be purified from natural sources, be produced using recombinant expression systems and optionally, purified, chemically synthesized, etc. Where appropriate, for example, in the case of chemically synthesized molecules, nucleic acids can comprise nucleoside analogues such as analogues having chemically modified bases or sugars, skeletal modifications, etc.

The term “nucleotide”, in the context of the present invention, refers to the monomers that make up nucleic acids. Nucleotides are made up of a pentose, a nitrogenous base and a phosphate group, and are bound by phosphodiester bonds. The nucleotides that are part of DNA and RNA differ in the pentose, this being deoxyribose and ribose respectively. Nitrogenous bases, in turn, are divided into purine nitrogenous bases, which are adenine (A) and guanine (G), and into pyrimidine nitrogenous bases, which are thymine (T), cytosine (C) and uracil (U). Thymine only appears in DNA, whereas uracil only appears in RNA.

“Cornea-SELEX” refers to Systematic Evolution of Ligands by Exponential Enrichment conducted on the cornea. SELEX is a laboratory technique used to screen a large pool of random nucleic acids or proteins to select ligands (such as nucleic acid aptamers or chemically modified nucleic acid aptamers) with high affinity and specificity for a particular target molecule. In “cornea-SELEX,” researchers use the cornea as the target, selecting ligands through iterative screening steps from DNA or RNA libraries that exhibit high affinity for specific receptors or molecules present on the cornea. This approach can be utilized to develop targeted drug delivery systems for the cornea, addressing challenges in the treatment of eye diseases.

### INDUSTRIAL APPLICABILITY

The aptamers obtained from cornea-SELEX can serve as a general ligand for ocular drug delivery, suggesting a promising avenue for the treatment of various eye diseases and even other diseases.

### REFERENCE

1 Ni S, Zhuo Z, Pan Y et al. Recent Progress in Aptamer Discoveries and Modifications for Therapeutic Applications. ACS Appl Mater Interfaces 2021; 13: 9500-9519.
2 Perez Tobia J, Huang P-J J, Ding Y et al. Machine Learning Directed Aptamer Search from Conserved Primary Sequences and Secondary Structures. ACS Synthetic Biology 2023; 12: 186-195.
3 Zhuo Z, Yu Y, Wang M et al. Recent Advances in SELEX Technology and Aptamer Applications in Biomedicine. Int J Mol Sci 2017; 18: 2142.
4 Musgrave C S A, Fang F. Contact Lens Materials: A Materials Science Perspective. Materials (Basel) 2019; 12: 261.

1. A targeted and slow-release drug conjugate composition, comprising a carrier conjugated with an aptamer-drug complex, wherein the aptamer-drug complex comprises at least one aptamer and at least one therapeutic agent, wherein the aptamer is a single-stranded oligonucleotides comprising a length ranging from 18-60 nucleotides.

2. The targeted and slow-release drug conjugate composition of claim 1, wherein the carrier is selected from the group consisting of microparticles, nanoparticles, microcapsules, microspheres, micelles, or liposomes.

3. The targeted and slow-release drug conjugate composition of claim 1, wherein the at least one therapeutic agent comprises ocular drugs comprising artificial tears, antibiotics, antivirals, anti-inflammatory drugs, glaucoma drugs, steroids, immunomodulators, or a combination thereof.

4. The targeted and slow-release drug conjugate composition of claim 1, wherein the at least one aptamer is further modified with a linker.

5. The targeted and slow-release drug conjugate composition of claim 1, wherein the linker comprises cholesterol, carbodiimide conjugation, thiol-maleimide crosslinking, or a combination thereof.

6. The targeted and slow-release drug conjugate composition of claim 1, wherein the single-stranded oligonucleotides comprise small molecules, proteins, peptides, or nucleic acids.

7. The targeted and slow-release drug conjugate composition of claim 1, wherein the targeted and slow-release drug conjugate composition is formulated as eye drops, eye ointment or eye gel, eye patches, eye capsules, eye film strips, ocular permeation agents, or ophthalmic gel.

8. An ocular device comprising an aptamer-drug complex attached to one or more ocular regions.

9. The ocular device of claim 8, wherein the ocular device is made from materials comprising poly methyl methacrylate (PMMA), polyvinyl alcohol (PVA), polyethylene glycol (PEG), dimethyl methacrylate (DMA), hydroxy ethyl methacrylate (HEMA), N-vinyl pyrrolidone (NVP), ethylene glycol dimethacrylate (EGDMA), poly dimethyl siloxane (PDMS), and 3-[tris(trimethyl siloxy)silyl]propyl methacrylate (TRIS).

10. The ocular device of claim 8, wherein the aptamer-drug complex exhibits strong binding with one or more ocular regions, characterized by a dissociation constant (K_{d}) of 1-5 nM.

11. The ocular device of claim 10, wherein the one or more ocular regions comprise an anterior segment comprising conjunctiva, cornea, sclera, iris, ciliary body, lens, or trabecular meshwork.

12. The ocular device of claim 10, wherein the one or more ocular regions comprise a posterior segment comprising retina, vitreous, choroid, optic nerve head, macula, subretinal space.

13. The ocular device of claim 8, wherein the ocular device comprises contact lenses, intraocular lenses.

14. A non-invasive, target-specific, and sustained drug delivery method applicable for the prevention and/or treatment of ocular diseases or disorders, including delivering a targeted drug conjugate composition to achieve precise release within one or more ocular tissues, wherein the method achieves release of at least 1-10% of the drug loaded per hour from biological materials.

15. The method of claim 14, wherein the one or more ocular tissues comprise an anterior segment comprising conjunctiva, cornea, sclera, iris, ciliary body, lens or trabecular meshwork.

16. The method of claim 14, wherein the one or more ocular tissues comprise a posterior segment comprising retina, vitreous humor, choroid, optic nerve head, macula, subretinal space.


SEQUENCE LISTING (37 sequences)

SEQ ID NO: 1 - DNA, 42 bp, synthetic construct
  source 1..42: mol_type = other DNA
gacgacggcaaggggaaagtggtcgtaatcacgacggtcgtc

SEQ ID NO: 2 - DNA, 42 bp, synthetic construct
  source 1..42: mol_type = other DNA
gacgacttatgcttgggacctgatccggactacggtgtcgtc

SEQ ID NO: 3 - DNA, 42 bp, synthetic construct
  source 1..42: mol_type = other DNA
gacgacgacaaaccagcggcttgtgagaatgcgttcgtcgtc

SEQ ID NO: 4 - DNA, 42 bp, synthetic construct
  source 1..42: mol_type = other DNA
gacgacgacaaaccagcggcttgtgagaatgcgttcgccgtc

SEQ ID NO: 5 - DNA, 42 bp, synthetic construct
  source 1..42: mol_type = other DNA
gacgacgacaaaccagcggcttgtgagaatgcggtcgtcgtc

SEQ ID NO: 6 - DNA, 30 bp, synthetic construct
  source 1..30: mol_type = other DNA
  modified_base 1..30: mod_base = OTHER; note = cholest-5-en-3beta-ol
gcagttgatcctttggataccctggttttt

SEQ ID NO: 7 - DNA, 21 bp, synthetic construct
  source 1..21: mol_type = other DNA
  modified_base 1..21: mod_base = OTHER; note = cholest-5-en-3beta-ol
acttcaacatctagctggtgg

SEQ ID NO: 8 - DNA, 93 bp, synthetic construct
  source 1..93: mol_type = other DNA
aatgatacggcgaccaccgagatctacactatcctctacactctttccctacacgacgctcttccgatctacagtcatcatagagtcgggacg

SEQ ID NO: 9 - DNA, 84 bp, synthetic construct
  source 1..84: mol_type = other DNA
caagcagaagacggcatacgagatgctcaggagtgactggagttcagacgtgtgctcttccgatctggaggctctcgggacgac

SEQ ID NO: 10 - DNA, 43 bp, synthetic construct
  source 1..43: mol_type = other DNA
gacgacaagttccccggcagggccattgtgagaaaacgtcgtc

SEQ ID NO: 11 - DNA, 42 bp, synthetic construct
  source 1..42: mol_type = other DNA
gacgacagcgagccggagggaatgagaacgagtggagtcgtc

SEQ ID NO: 12 - DNA, 42 bp, synthetic construct
  source 1..42: mol_type = other DNA
gacgactctactttacatggtcaatgggcgtgcggtgtcgtc

SEQ ID NO: 13 - DNA, 21 bp, synthetic construct
  source 1..21: mol_type = other DNA
cctgtcctgcgtgttgaaaga

SEQ ID NO: 14 - DNA, 20 bp, synthetic construct
  source 1..20: mol_type = other DNA
gggaactgggcagactcaaa

SEQ ID NO: 15 - DNA, 20 bp, synthetic construct
  source 1..20: mol_type = other DNA
cacagacagccactcacctc

SEQ ID NO: 16 - DNA, 20 bp, synthetic construct
  source 1..20: mol_type = other DNA
ttttctgccagtgcctcttt

SEQ ID NO: 17 - DNA, 24 bp, synthetic construct
  source 1..24: mol_type = other DNA
tttcagagacagcagagcacacaa

SEQ ID NO: 18 - DNA, 23 bp, synthetic construct
  source 1..23: mol_type = other DNA
cacacagagctgcagaaatcagg

SEQ ID NO: 19 - DNA, 19 bp, synthetic construct
  source 1..19: mol_type = other DNA
gaaggtgaaggtcggagtc

SEQ ID NO: 20 - DNA, 20 bp, synthetic construct
  source 1..20: mol_type = other DNA
gaagatggtgatgggatttc

SEQ ID NO: 21 - DNA, 73 bp, synthetic construct
  source 1..73: mol_type = other DNA
ggaggctctcgggacgacnnnnnnnnnnnnnnnnnnnnnnnnnnnnnngtcgtcccgactctatgatgactgt

SEQ ID NO: 22 - DNA, 18 bp, synthetic construct
  source 1..18: mol_type = other DNA
  modified_base 1..18: mod_base = OTHER; note = 3'-biotin
gtcgtcccgagagccata

SEQ ID NO: 23 - DNA, 18 bp, synthetic construct
  source 1..18: mol_type = other DNA
ggaggctctcgggacgac

SEQ ID NO: 24 - DNA, 23 bp, synthetic construct
  source 1..23: mol_type = other DNA
acagtcatcatagagtcgggacg

SEQ ID NO: 25 - DNA, 23 bp, synthetic construct
  source 1..23: mol_type = other DNA
  modified_base 1..23: mod_base = OTHER; note = 5'-biotin
acagtcatcatagagtcgggacg

SEQ ID NO: 26 - DNA, 47 bp, synthetic construct
  source 1..47: mol_type = other DNA
  modified_base 1..47: mod_base = OTHER; note = cholest-5-en-3beta-ol
gacgacggcaaggggaaagtggtcgtaatcacgacggtcgtcttttt

SEQ ID NO: 27 - DNA, 47 bp, synthetic construct
  source 1..47: mol_type = other DNA
  modified_base 1..47: mod_base = OTHER; note = cholest-5-en-3beta-ol
gacgacttatgcttgggacctgatccggactacggtgtcgtcttttt

SEQ ID NO: 28 - DNA, 30 bp, synthetic construct; identical to SEQ ID NO: 6
  source 1..30: mol_type = other DNA
  modified_base 1..30: mod_base = OTHER; note = cholest-5-en-3beta-ol

SEQ ID NO: 29 - DNA, 21 bp, synthetic construct
  source 1..21: mol_type = other DNA
  modified_base 1..21: mod_base = OTHER; note = cholest-5-en-3beta-ol
acttcaacatctagctggtgg

SEQ ID NO: 30 - DNA, 42 bp, synthetic construct; identical to SEQ ID NO: 1
  source 1..42: mol_type = other DNA
  modified_base 1..42: mod_base = OTHER; note = 3-end-6-Carboxyfluorescein

SEQ ID NO: 31 - DNA, 42 bp, synthetic construct; identical to SEQ ID NO: 2
  source 1..42: mol_type = other DNA
  modified_base 1..42: mod_base = OTHER; note = 3-end-6-Carboxyfluorescein

SEQ ID NO: 32 - DNA, 42 bp, synthetic construct; identical to SEQ ID NO: 1
  source 1..42: mol_type = other DNA
  modified_base 1..42: mod_base = OTHER; note = 5'-biotin

SEQ ID NO: 33 - DNA, 42 bp, synthetic construct; identical to SEQ ID NO: 2
  source 1..42: mol_type = other DNA
  modified_base 1..42: mod_base = OTHER; note = 5'-biotin

SEQ ID NO: 34 - DNA, 20 bp, synthetic construct
  source 1..20: mol_type = other DNA
gtcttcctctctcacgcacc

SEQ ID NO: 35 - DNA, 20 bp, synthetic construct
  source 1..20: mol_type = other DNA
tgggctagaggcttgtcact

SEQ ID NO: 36 - DNA, 20 bp, synthetic construct
  source 1..20: mol_type = other DNA
gtcttcctctctcacgcacc

SEQ ID NO: 37 - DNA, 20 bp, synthetic construct
  source 1..20: mol_type = other DNA
tgggctagaggcttgtcact